(19) United States
(12) Patent Application Publication
(10) Pub. No.: US 2003/0087887 A1
(43) Pub. Date: May 8, 2003

(54) Novel fusidic acid derivatives

(76) Inventor: Tore Duvold, Frederiksberg (DK)

Correspondence Address: BIRCH STEWART KOLASCH & BIRCH, PO BOX 747, FALLS CHURCH, VA, 22040-0747, US

(21) Appl. No.: 10/100,992

(22) Filed: Mar. 21, 2002

Related U.S. Application Data
(60) Provisional application No. 60/299,831, filed on Mar. 21, 2001.

(51) Int. Cl. A61K 31/56; C07J 41/00; C07J 9/00

(52) U.S. Cl. 514/182; 552/540; 552/515; 552/520; 552/521

(57) ABSTRACT

Compounds of the general formula I

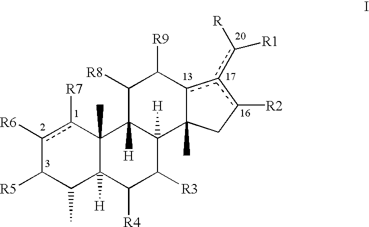

wherein

R₁ is hydrogen, halogen, CH₃, CH₂—OH, COOH, CH₂—OSO₃, CH₂—NH(CH₂)ₐ—R₁₀, or C(═O)—NH—(CH₂)ₐ—R₁₀ wherein R₁₀ is —NH₂, —NH—(CH₂)_{b}—NH₂, —NH—(CH₂)_{b}—NH—(CH₂)_{c}—NH₂, —NH—(CH₂)_{b}—NH—(CH₂)_{c}—NH—(CH₂)_{d}—NH₂, —NH—(CH₂)_{b}—NH—(CH₂)_{c}—NH—(CH₂)_{d}—NH(CH₂)ₑ—NH₂, —NH—(CH₂)_{b}—NH—(CH₂)O—NH—(CH₂)_{d}—NH—(CH₂)ₑ—NH—(CH₂)_{f}—NH₂, a saturated or unsaturated heterocyclic ring comprising 1 or 2 heteroatoms, or —NH—(CH₂)_{b}—R₁₁, wherein R₁₁ is a saturated or unsaturated heterocyclic ring comprising 1 or 2 heteroatoms, and a, b, c, d, e and f are the same or different and individually represent integers of from 1 to 5;

R₂ is hydrogen, halogen, —OH or —OR₁₂, wherein R₁₂ is SO₃, C₁₋₆ alkyl or C₁₋₆ acyl, —NH—(CH₂)ₐ—R₁₀;

R is hydrogen, halogen, a lipophilic group, —NH₂—(CH₂)ₐ—R₁₀ or CH₂—NH—(CH₂)ₐ—R₁₀;

R₄, R₅, R₆, R₇ and R₉ are the same or different and individually represent hydrogen, halogen, —OH, —OSO₃ or —NH—(CH₂)ₐ—R₁₀;

R₃ and R₈ are the same or different and individually represent hydrogen, halogen, —OH or OSO₃; and

the dotted lines between carbon atoms 1 and 2, 13 and 17, 16 and 17, and 17 and 20 indicate the presence of a single or double bond;

provided that at least one and not more than two of R, R₁, R₂, R₄, R₅, R₆, R₇ or R₉ is —NH—(CH₂)ₐ—R₁₀, CH₂—NH—(CH₂)ₐ—R₁₀ or C(═O)—NH—(CH₂)ₐ—R₁₀, and the others are hydrogen, —OH or —OSO₃, or (for R₂)—OR₁₂; and further provided that at least one and not more than four of R₂—R₉ are —OH or —OSO₃;

and pharmaceutically acceptable salts and esters thereof are active against a broad spectrum of microorganisms, and may therefore be used in the treatment of microbial infections.

### FIELD OF INVENTION

The present invention relates to novel polyaminated fusidic acid derivatives with a broad spectrum of antimicrobial activity, as well as the use of the compounds in therapy, in particular as anti-infective agents.

### BACKGROUND OF THE INVENTION

In the field of antibiotics, drug resistance is an ever-increasing problem posing a serious threat to public health. The general belief for many years that infectious diseases could be controlled by the current arsenal of antibacterial drugs has resulted in the development of new and more efficient drugs getting a low priority. Recent widespread emergence of multiple resistance among pathogenic bacteria has sparked renewed interest in the discovery of new antibiotics. Although resistance to many antibiotics such as lactams, macrolides, tetracyclines and aminoglycosides, and the rapid spread of resistance have been recognised for many years, it was assumed that reserve drugs like glycopeptides and fluoroquinolones were sufficient to combat most infections. However, the many alarming reports of vancomycin-resistance, multiple drug resistance and examples of transfer of resistance genes between different species in the late 1980s and early 1990s has brought the issue of drug resistance to the attention of health authorities and the pharmaceutical industry.

Fusidic acid belongs to the fusidanes which is a small family of naturally occurring antibiotics having in common a tetracyclic ring system with the unique chair-boat-chair conformation separating them from regular steroids. The fusidanes therefore do not exert any hormonal activity. Fusidic acid, a fermentation product of Fusidium coccineum, is the most active compound of the fusidanes and is the only compound used clinically in treatment of infectious diseases. Fusidic acid (Fucidin®) is used clinically for the treatment of severe staphylococcal infections, particularly in bone and joint infections, in both the acute and the intractable form of the disease (Kuchers et al., 1997 and references cited therein). It is generally given in combination with common antibiotics such as penicillins, erythromycins or clindamycin. It has also been used as an alternative to vancomycin in the control of Clostridium difficile.

Fusidic acid is widely used in local therapy for a number of skin and eye infections caused by staphylococci. Compared to staphylococci, all other gram-positive cocci are much less susceptible to fusidic acid. Several streptococci including multi-resistant strains of Streptococcus pneumoniae are only partly resistant to fusidic acid. Other sensitive bacteria include gram-positive anaerobic cocci, such as Peptococcus and Peptostreptococcus spp., aerobic or anaerobic gram-positive bacteria, such as Corynebacterium diphtheriae, Clostridium tetani, Clostridium difficile and Clostridium perfingens. Gram-negative bacteria are resistant except for Neisseria spp. and Legionella pneumophila. The drug is highly potent against both intracellular and extracellular M. leprae.

Fusidic acid exerts its antibacterial activity by blocking bacterial protein synthesis through inhibition of translocation of the ribosome relative to niRNA through interference with the “G” factor (EF-G). The exact mechanism of action is being studied on a molecular level but is so far not completely understood (Laurberg et al., 2000). The difference in the mode of action of the drug explains the absence of cross-resistance between fusidic acid and common antibiotics such as penicillins and cephalosporins.

More recently, a steroidal antibiotic was isolated from the stomach of the dogfish shark, Squalus acanthias (Moore et al., 1993; Rao et al., 2000). The compound, which is based on a steroid backbone comprising a polyamine and sulphate functionality, was termed squalamine and was found to have broad-spectred antibiotic properties against gram-positive and gram-negative bacteria, fungi and protozoa. The use of native squalamine as an antimicrobial agent is disclosed in U.S. Pat. No. 5,192,756. Squalamine has also been prepared by chemical synthesis although the procedure has been found to be rather cumbersome. A number of squalamine analogues and their use as antibiotics are disclosed in WO 00/09137.

Motivated by the serious difficulties in obtaining squalamine from natural sources as well as by chemical synthesis, mimics based on cholic acid derivatives have been prepared (Sadownik, et al., 1995; Kikuchi et al., 1997; Savage and Li, 2000; Hong-Seok 2000). Some of these analogues have shown broad antibacterial activity, both against gram-negative and gram-positive bacteria. In addition, some of the active analogues have a very interesting synergistic effect with some known polar antibiotics. This may partly be due to assisted transport over the bacterial cell membrane by the squalamine mimic. One of the main, and probably the crucial, characteristic of the squalamine mimics having a cholic acid backbone is their relatively polar system on one surface of the steroid framework and a hydrophobic surface and a positively charged side chain on the other surface, a so-called facial amphiphile.

### SUMMARY OF THE INVENTION

Compared to other antibiotics, fusidic acid has so far not developed serious problems with drug resistance. However, the substance in itself has a fairly limited antibiotic spectrum, and it might therefore be desirable to develop novel analogues based on the fusidic acid backbone, but comprising other pharmacophores than those present in the native molecule so as to exhibit an increased antibiotic actitvity against a broader range of pathogenic microorganisms. An attractive option might therefore be to prepare fusidic acid derivatives comprising a fusidic acid steroid backbone and a side chain derived from a linear polyamine, e.g. a spermine or spermidine chain of squalamine with a view to developing fusidic acid derivatives with much wider antibacterial spectrum having no cross-resistance with other clinically used antibiotics and preferably with a bactericidal action.

Accordingly, the present invention relates to a compound of the general formula I

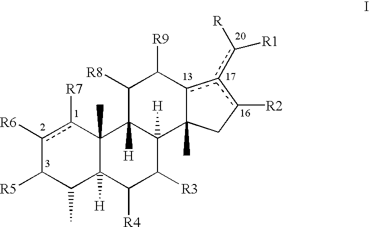

wherein

R₁ is hydrogen, halogen, CH₃, CH₂—OH, COOH, CH₂—OSO₃, CH₂—NH—(CH₂)ₐ—R₁₀, or C(═O)—NH—(CH₂)ₐ—R₁₀ wherein R₁₀ is —NH₂, —NH—(CH₂)_{b}—NH₂, —NH—(CH₂)_{b}—NH—(CH₂)_{c}—NH₂, —NH—(CH₂)_{b}—NH—(CH₂)_{c}—NH—(CH₂)_{d}—NH₂, —N—(CH₂)_{b}—NH—(CH₂)_{c}—NH—(CH₂)_{d}—NH—(CH₂)ₑ—NH₂—NH—(CH₂)_{b}—NH—(CH₂)_{c}—NH—(CH₂)_{d}—NH—(CH₂)ₑ—NH—(CH₂)_{f}—NH₂, a saturated or unsaturated heterocyclic ring comprising 1 or 2 heteroatoms, or —NH—(CH₂)_{b}—R₁₁, wherein R₁₁ is a saturated or unsaturated heterocyclic ring comprising 1 or 2 heteroatoms, and a, b, c, d, e and f are the same or different and individually represent integers of from 1 to 5;

R₂ is hydrogen, halogen, —OH or —OR₁₂, wherein R₁₂ is SO₃, C₁₋₆ alkyl or C₁₋₆ acyl, —NH—(CH₂)ₐ—R₁₀;

R is hydrogen, halogen, a lipophilic group, —NH₂(CH₂)ₐ—R₁₀ or CH₂—NH—(CH₂)ₐ—R₁₀;

R₄, R₅, R₆, R₇ and R₉ are the same or different and individually represent hydrogen, halogen, —OH, —OSO₃ or —NH—(CH₂)ₐ—R₁₀;

R₃ and R₈ are the same or different and individually represent hydrogen, halogen, —OH or OSO₃; and

the dotted lines between carbon atoms 1 and 2, 13 and 17, 16 and 17, and 17 and 20 indicate the presence of a single or double bond;

provided that at least one and not more than two of R, R₁, R₂, R₄, R₅, R₆, R₇ or R₉ is —NH—(CH₂)ₐ—R₁₀, CH₂—NH—(CH₂)ₐ—R₁₀ or C(=O)—NH—(CH₂)ₐ—R₁₀, and the others are hydrogen, —OH or —OSO₃, or (for R₂)—OR₁₂; and further provided that at least one and not more than four of R₂—R₉ are —OH or —OSO₃;

and pharmaceutically acceptable salts and esters thereof.

Compounds of formula I have been found to exert antimicrobial activity across a much broader range of microorganisms than fusidic acid, including activity against gram-positive bacteria such as Streptococcus pyrogenes, Staphylococcus aureus, including multidrug resistant strains, and Staphylococcus epidermidis, gram-negative bacteria such as Pseudomonas and Escherichia coli, yeast such as Candida albicans and Saccharomyces cerevisiae and fungi such as Aspergillus flavus and Aspergillus niger. The level of activity is equal to or better than that reported for naturally occurring squalamines (Moore et a[, 1993; Kikuchi et al., 1997; Rao et al., 2000) and the most potent of the known squalamine mimics, SM-7 (Kikuchi et al., 1997).

The exact mechanism of action of the present compounds is currently unknown. Without wishing to be limited to a particular hypothesis, it is believed that they may perforate cell membranes, and that membrane lysis could occur through pore formation. In this way, the present compounds may be able to circumvent two major drug resistance mechanisms, i.e.enzymatic degradation in the cell and export <pathways (Sadownik et al., 1995; Savage and Li, 2000 and references cited therein).

In another aspect, the invention relates to a pharmaceutical composition comprising a compound of formula I together with a pharmaceutically acceptable excipient or diluent, and to the use of compounds of formula I as medicaments.

In a further aspect, the invention relates to the use of a compound of formula I in the manufacture of a medicament for the prevention or treatment of infection.

In a still further aspect, the invention relates to a method of preventing or treating infection, the method comprising administering to a patient in need thereof an effective amount of a compound of formula I.

### DETAILED DESCRIPTION OF THE INVENTION

Definitions

In the present context, the term “alkyl” is intended to indicate a univalent radical derived from straight or branched alkane by removing a hydrogen atom from any carbon atom. The term includes the subclasses primary, secondary and tertiary alkyl, such as methyl, ethyl, n-propyl, isopropyl, n-butyl, isobutyl, sec.-butyl, tert.-butyl, n-pentyl, isopentyl, n-hexyl and isohexyl.

The term “alkoxy” is intended to indicate a radical of formula OR′, wherein R′ is alkyl as defined above, e.g. methoxy, ethoxy, propoxy, butoxy, etc.

The term “alkoxycarbonyl” is intended to indicate a radical of formula COOR′ wherein R′ is alkyl as defined above, e.g. methoxycarbonyl, ethoxycabonyl, n-propoxycarbonyl, isopropoxycarbonyl, etc.

The term “cycloalkyl” is intended to indicate a saturated cycloalkane radical, e.g. cyclopropyl, cyclobutyl, cyclopentyl or cyclohexyl.

The term “cycloalkenyl” is intended to indicate monounsaturated cyclic hydrocarbon radicals, e.g. cyclopropenyl, cyclobutenyl, cyclopentenyl or cyclohexenyl.

The term “aryl” is intended to include radicals of carbocyclic aromatic rings, in particular 5- or 6-membered rings, optionally fused bicyclic rings, e.g. phenyl or naphthyl. The term “heteroaryl” is intended to include radicals of heterocyclic aromatic rings, in particular 5- or 6-membered rings with 1-3 heteroatoms selected from 0, S and N, or optionally fused bicyclic rings with 1-4 heteroatoms, e.g. pyridyl, tetrazolyl, thiazolyl, imidazolyl, pyrazolyl, oxazolyl, isoxazolyl, thienyl, pyrazinyl, isothiazolyl, benzimidazolyl and benzofuranyl.

The term “saturated or unsaturated heterocyclic ring comprising 1 or 2 hetero atoms “is intended to indicate heteroaryl, as defined above, and compounds such as pyrrolidinyl, pyrrolinyl, imidazolidinyl, pirazolidinyl, piperidyl, piperazinyl, morpholinyl, tetrahydropyranyl, tetrahydrofuranyl.

The term “acyl” refers to a radical of formula R′—CO—, wherein R′ is alkyl as indicated above.

The term “aralkyl” is intended to indicate an aromatic ring with an alkyl side chain, e.g. benzyl.

The term “halogen” is intended to indicate fluoro, chloro, bromo or iodo.

The term “polyamine building block” is intended to indicate compounds of the formula H₂N—(CH₂)ₐ—R₁₀ or H₂N—(CH₂)ₐ—NH—(CH₂)_{b}—R₁₁, wherein a, b, R₁₀ and R₁₁ are as defined for formula I.

The term “pharmaceutically acceptable salt” is intended to indicate alkali metal or alkaline earth metal salts, for instance sodium, potassium, magnesium or calcium salts, as well as silver salts and salts with bases such as ammonia or suitable non-toxic amnines, e.g. lower alkylamines, for instance triethylamine, hydroxy-lower alkylamines, for instance 2-hydroxyethylamine or bis-(2-hydroxyethyl)amine, cycloalkylamines, for instance dicyclohexylamine, or benzylamines, such as N,N′-dibenzylethylenediamine and dibenzylamine, as well as salts with suitable organic or inorganic acids, such as hydrochloric, hydrobromic, hydroiodic, sulfuric, nitric, phosphoric, acetic, lactic, maleic, phtalic, citric, propionic, benzoic, glutaric, gluconic, metanesulfonic, salicylic, succinic, tartaric, toluenesulfonic, sulfamic or fumaric acid.

The term “pharmaceutically acceptable esters” is intended to indicate easily hydrolysable esters such as alkanoyloxyalkyl, aralkanoyloxyalkyl, aroyloxyalkyl, e.g. acetoxymethyl, pivaloyloxymethyl, benzoyloxymethyl esters and the corresponding 1′-oxyethyl derivatives, or alkoxycarbonyloxyalkyl esters, e.g. methoxycarbonyloxymethyl esters and ethoxycarbonyloxymethyl esters and the corresponding 1′-oxyethyl derivatives, or lactonyl esters, e.g. phthalidyl esters, or dialkylaminoalkyl esters, e.g. dimethylaminoethyl esters. Easily hydrolysable esters include in vivo hydrolysable esters of the compounds of formula I. Such esters may be prepared by conventional methods known to persons skilled in the art, such as method disclosed in GB patent No. 1 490 852 incorporated herein by reference.

Preferred embodiments of the compound of formula I Preferred compounds of formula I are compounds of formula Ia

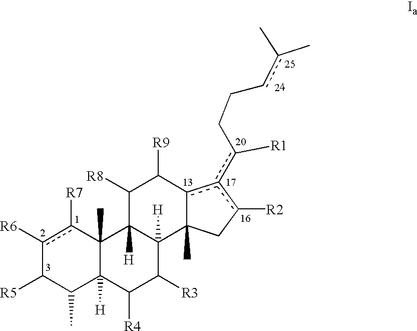

wherein

R₁ is CH₃, CH₂—NH—(CH₂)ₐ—R₁₀ or C(═ONH(CH₂)ₐ—R₁₀, wherein R₁₀ and a are as indicated above; R₂ and R₅ are hydrogen, —OH or —OSO₃, or, for R₂, —OR₁₂, wherein R₁₂ is as indicated above; R₃, R₄, R₆, R₅ and R₉ are hydrogen, —OH or —OSO₃; and the dotted line between carbon atoms 1 and 2, 13 and 17, 16 and 17, 17 and 20, 24 and 25 indicates the presence of a single or double bond;

provided that at least one and not more than four of R₂, R₃, R₄, R₅, R₆, R₈ and R₉ are H or OSO₃.

It is currently believed that the present fusidic acid derivatives having a lipophilic sterol backbone may form an intramolecular hydrogen bond (R₅═OH) or salt bridge (R₅═OSO₃) between the cationic terminus of the polyamine side chain and a hydroxy or sulphate group elsewhere in the molecule (analogous to that shown for squalamine mimics in Kikuchi et aL, 1997 supra, and references cited therein), thus imparting a circular conformation to the molecule. It is therefore preferred that at least one of R₂, R₃, R₄, R₄, R₅, R₆, R₈ or R₉ is —OH or —OSO₃ so as to make bridge formation possible. The relative positions of the polyamine side chain and the sulphate group are also thought to be important for the activity and/or potency of the compounds. When the polyamine side chain is located in position R₁, the —OH or —OSO₃ group is preferably located in position Rs so that the molecule is brought into the desired circular (active) conformation.

There are several chiral centres in the compounds according to the invention because of the presence of asymmetric carbon atoms. The presence of several asymmetric carbon atoms gives rise to a number of stereoisomers with R or S configuration at each chiral centre. General formula I and Ia, and (unless specified otherwise) all other formulae in this specification are to be understood to include all such stereoisomers in pure form and as mixtures (for example stereoisomeric mixtures) except where the configuration is expressly indicated.

In the formulas herein plain lines depict bonds which may be above or below the plane of the drawing; bonds to atoms above the plane are shown with a bold wedge starting from an atom in the plane of the drawing at the narrow end of the wedge; and bonds to atoms below the plane are shown with short parallel (wedged) lines. Substituents above the plane are described as 0 and shown as a bold wedge, those below the plane are described as a and shown by a line with short parallel (wedged) lines. In the compounds of formula I and Ia, it would appear that a 17a side chain is more favourable than the 17β configuration which could be due to a preferred circular conformation of the active compound.

In preferred embodiments of the compounds of the invention, a is 2 or 3. R₁₀ is preferably —NH—(CH₂)_{b}—NH₂, wherein b has the meaning indicated above, in particular 3 or 4.

R₁₀ may also be —NH—(CH₂)_{b}—NH—(CH₂)_{c}—NH₂, wherein b and c are as indicated above, in particular wherein c is 2 or 3.

In a further preferred embodiment, R₁₀ is —NH—(CH₂)_{b}—NH—(CH₂)_{c}—NH—(CH₂)_{d}—NH₂, wherein b, c and d are as indicated above, in particular wherein d is 2, 3 or 4.

In a still further embodiment, R₁₀ may be —NH—(CH₂)_{b}—NH—(CH₂)_{c}—NH—(CH₂)_{d}—NH—(CH₂)ₑ—NH₂, wherein b, c, d and e are as indicated above, in particular wherein e is 2, 3 or 4.

In specific embodiments, RI may be CH₂—NH—(CH₂)₃—NH—(CH₂)₄—NH—(CH₂)₃—NH₂, —NH—(CH₂)₃—NH—(CH₂)₄—NH—(CH₂)₃—NH₂, or C(═O)—NH—(CH₂)₃—NH—(CH₂)₄—NH—(CH₂)₃—NH₂.

R₂ in formula I or Ia is preferably hydrogen or —OR₁₂, wherein R₁₂ is C₁₋₆ alkyl or C₁₋₆ acyl, or wherein R₁₂ is —NH—(CH₂)ₐ—R₁₀, CH₂—NH—(CH₂)ₐ—R₁₀ or C(═O)—NH—(CH₂)ₐ—R₁₀, wherein R₁₀ and a are as indicated above.

In formula I or Ia, R₃, R₅ and/or R₈ are preferably an —OH group. In particular, R₅ and R₈ are both an —OH group, or R₅ may in addition be a —OSO₃ group.

In the compounds of formula I, R is preferably a lipophilic group, i.e. a group which is predominantly non-polar. Such a group is present in native fusidic acid at this position and may be of significance to the ability of the compound to lodge in cell membranes which are also lipophilic in nature. Examples of such lipophilic groups are branched or straight C₁₋₁₀ alkyl, aryl or C₃₋₈ cycloalkyl, C₃₋₈ cycloalkenyl and aralkyl with 1-10 carbon atoms in the alkyl moiety, C₁₋₁₀ alkylaryl, C₁₋₁₀ alkyl-C₃₋₈ cycloalkyl, C₁₋₁₀ alkyl-C₃₋₈ cycloalkenyl, C₁₋₁₀ alkoxy or heteroaryl. Preferably, the lipophilic group R is the side chain found in native fusidic acid (as shown in formula Ia), or a closely related alkyl group.

Examples of compounds of the present invention are selected from the group consisting of

21-N-{3′-aminopropyl}-fusid-21-amide (Compound 101),

21-N-{2′-[(2′-aminoethyl)amino]ethyl}-fusid-21-amide (Compound 102),

21-N-{3′-[3′-aminoproyl)amino]propyl}-fusid-21-amide (Compound 103),

21-N-{3′-[(4′-aminobutyl)amino]propyl}-fusid-21-amide (Compound 104),

21-N-[2′-({3′-[(2′-aminoethyl)amino]propyl}amino)ethyl]-fusid-21-amide (Compound 105),

21-N-[3′-({3′-[(3′-aminopropyl)amino]propyl}amino)propyl]-fusid-21-amide (Compound 106),

21-N-[3′-({4′-[(3′-aminopropyl)amino]butyl}amino)propyl]-fusid-21-amide (Compound 107),

21-N-[3′-({2′-[(3′-aminopropyl)amino]ethyl}amino)propyl]-fusid-21-amide (Compound 108),

21-N-[4′-({3′-[(4′-aminobutyl)amino]propyl}amino)butyl]-fusid-21-amide (Compound 109),

21-N-{6′-[(6′-aminohexyl)amino]hexyl}-fusid-21-amide (Compound 110),

21-N-{8′-[(8′-aminooctyl)amino]octyl}-fusid-21-amide (Compound 111),

21-N-(2′-{[2′-({2′-[(2′-aminoethyl)amino]ethyl)amino)ethyl}amino}ethyl)-fusid-21-amide (Compound 112),

3-N-[3′-({3′-[(3′-aminopropyl)amino]propyl}amino)propyl]-fusidic acid (Compound 113)

3-N-[2′-({3′-[(2′-aminoethyl)amino]propyl}amino)ethyl]-fusidic acid (Compound 114)

3-N-[3′-({2′-[(3′-aminopropyl)amino]ethyl}amino)propyl]fusidic acid (Compound 115)

21-N-{2′-({2′-[(2′-aminoethyl)amino]ethyl}amino)ethyl)amino}-17R,20S,24,25-tetrahydrofusid-21-amide (Compound 116),

21-N-{2′-[(2′-aminoethyl)amino]ethyl}-17R,20S,24,25-tetrahydrofusid-21-amide (Compound 117),

21-N-{6′-aminohexyl}-17R,20S,24,25-tetrahydrofisid-21-amide (Compound 118),

21-N-{3′-aminopropyl}-17R,20S,24,25-tetrahydrofusid-21-amide (Compound 119),

21-N-{3′-[3′-aminoproyl)amino]propyl}-17R,20S,24,25-tetrahydrofusid-21-amide (Compound 120),

21-N-{4′-[(3′-aminopropyl)amino]butyl}-17R,20S,24,25-tetrahydrofusid-21-amide (Compound 121),

21-N-[2′-({3′-[(2′-aminoethyl)amino]propyl}amino)ethyl]-17R,20S,24,25-terahydrofusid-21-amide (Compound 122),

21-N-[4′-({3′-[(4′-aminobutyl)amino]propyl}amino)butyl]-17R,20S,24,25-tetrahydrofasid-21-amide (Compound 123),

21-N-[3′-({2′-[(3′-aminopropyl)amino]ethyl}amino)propyl]-17R,20S,24,25-tetrahydrofusid-21-amide (Compound 124),

21-N-[3′-({4′-[(3′-aminopropyl)amino]butyl}amino)propyl]-17R,20S,24,25-tetrahydrofusid-21-amide (Compound 125),

21-N-{6′-[(6′-aminohexyl)amino]hexyl}-17R,20S,24,25-tetrahydrofusid-21-amide (Compound 126),

15, 21-N-{8′-[(8′-aminooctyl)amino]octyl}-17R,20S,24,25-tetrahydrofusid-21-amide (Compound 127),

21-N-[3′-({3′-[(3′-aminopropyl)amino]propyl}amino)propyl]-17R,20S,24,25-tetrahydrofusid-21-amide (Compound 128),

21-N-(2′-{[2′-({2′-[(2′-aminoethyl)amino]ethyl}amino)ethyl]amino}ethyl)-17R,20S,24,25-tetrahydrofusid-21-amide (Compound 129),

21-N-{6′-aminohexyl}-17R,20S,24,25-tetrahydrofusid-21-amide (Compound 130),

21-N-{3′-[3′-aminoproyl)amino]propyl}-17R,20S,24,25-tetrahydrofusid-21-amide (Compound 131),

21-N-{2′-[(2′-aminoethyl)amino]ethyl}-17R,20S,24,25-tetrahydrofusid-21-amide (Compound 132),

21-N-{2′-({3′-[(2′-aminoethyl)amino]propyl}amino)ethyl}-17R,20S,24,25-tetrahydrofusid-21-amide (Compound 133),

21-N-{2′-({2′-[(2′-aminoethyl)amino]ethyl}amino)ethyl)amino}-17R,20S,24,25-tetrahydrofusid-21-amide (Compound 134),

21-N-[3′-({4′-[(3′-aminopropyl)amino]butyl}amino)propyl]-17R,20S,24,25-tetrahydrofusid-2 1-amide (Compound 135),

21-N-(2′-{[2′-({2′-[(2′-aminoethyl)amino]ethyl}amino)ethyllamino}ethyl)-17R,20S,24,25-tetrahydrofusid-21-amide (Compound 136),

21-N-{4′-[(3′-aminopropyl)amino]butyl}-17R,20S,24,25-tetrahydrofusid-21-amide (Compound 137),

21-N-{[4′-({3′-[(4′-aminobutyl)amino]propyl}amino)butyl]}-17R,20S,24,25-tetrahydrofusid-21-amide (Compound 138),

21-N-[3′-({3′-[(3′-aminopropyl)amino]propyl}amino)propyl]-16(17)-en-17,20,24,25-tetrahydrofusidan-21-carboxamide (Compound 139),

21-N-{6′-aminohexyl}-16-desacetoxy-17R,20S,24,25-tetrahydrofusid-21-amide (Compound 140),

21-N-{6′-aminohexyl}-16-desacetoxy-17R,20S,24,25-tetrahydrofusid-21-amide (Compound 141), (C-20 epimer of Compound 140),

21-N-{2′-[(2′-aminoethyl)amino]ethyl}-16-desacetoxy-17R,20S,24,25-tetrahydrofisid-21-amide (Compound 142),

21-N-{3′-[3′-aminoproyl)amino]propyl}-16-desacetoxy-17R,20S,24,25-tetrahydrofusid-21-amide (Compound 143),

21-N-{3′-[(4′-aminobutyl)amino]propyl}-16-desacetoxy-17R,20S,24,25-tetrahydrofusid-21-amide (Compound 144),

21-N-{[3′-({3′-[(3′-aminopropyl)amino]propyl}amino)propyl]}-16-desacetoxy-17R,20S,24,25-tetrahydrofusid-21-amide (Compound 145),

21-N-{[3′-({4′-[(3′-aminopropyl)amino]butyl}amino)propyl]}-16-desacetoxy-17R,20S,24,25-tetrahydrofusid-21-amide (Compound 146),

21-N-(2′-{[2′-({2′-[(2′-aminoethyl)amino]ethyl}amino)ethyl]amino}ethyl)-16-desacetoxy-17R,20S,24,25-tetrahydrofusid-21-amide (Compound 147),

21-N-{2′-({2′-[(2′-aminoethyl)amino]ethyl}amino)ethyl)amino}-16-desacetoxy-17R,20S,24,25-tetrahydrofusid-2 1-amide (Compound 148),

21-N-[2′-({3′-[(2′-aminoethyl)amino]propyl}amino)ethyl]-16-desacetoxy-17R,20S,24,25-tetrahydrofusid-21-amide (Compound 149),

21-N-{6′-aminohexyl}-11-desoxy-16-desacetoxy-17S,20,24,25-tetrahydrofusid-21-amide (Compound 150),

21-N-{3′-[3′-aminoproyl)amino]propyl}-11-desoxy-16-desacetoxy-17S,20,24,25-tetrahydrofusid-21-amide (Compound 151),

21-N-[2′-({3′-[(2′-aminoethyl)amino]propyl}amino)ethyl]-11-desoxy-16-desacetoxy-17S,20,24,25-tetrahydrofusid-21-amide (Compound 152),

21-N-[3′-({2′-[(3′-aminopropyl)amino]ethyl}amino)propyl]-11-desoxy-16-desacetoxy-17S,20,24,25-tetrahydrofusid-21-amide (Compound 153),

21-N-{4′-[(3′-aminopropyl)amino]butyl}-11-desoxy-16-desacetoxy-17S,20,24,25-tetrahydrofusid-21-amide (Compound 154),

21-N-[3′-({3′-[(3′-aminopropyl)aniinolpropyl}amino)propyl]-11-desoxy-16-desacetoxy-17S,20,24,25-tetrahydrofusid-21-amide (Compound 155),

21-N-[3′-({4′-[(3′-aminopropyl)amino]butyl}amino)propyl]-11-desoxy-16-desacetoxy-17S,20,24,25-tetrahydrofusid-21-amide (Compound 156),

21-N—[4′-({3′-[(4′-aminobutyl)amino]propyl}amino)butyl]-11-desoxy-16-desacetoxy-17S,20,24,25-tetrahydrofusid-21-amide (Compound 157),

21-N-{6′-aminohexyl}-3β-desacetoxy-17R,20S,24,25-tetrahydrofusid-21-amide (Compound 158),

21-N-{2′-[(2′-aminoethyl)amino]ethyl}-3β-desacetoxy-17R,20S,24,25-tetrahydrofusid-21-amide (Compound 159),

21-N-{3′-[3′-aminoproyl)amino]propyl}-3β-desacetoxy-17R,20S,24,25-tetrahydrofusid-21-amide (Compound 160),

21-N-{3′-[(4′-aminobutyl)amino]propyl}-3β-desacetoxy-17R,20S,24,25-tetrahydrofusid-21-amide (Compound 161),

21-N-[2′-({3′-[(2′-aminoethyl)amino]propyl}amino)ethyl]-3β-desacetoxy-17R,20S,24,25-tetrahydrofusid-21-amide (Compound 162),

21-N-[3′-({3′-[(3′-aminopropyl)aminojpropyl}amino)propyl]-3β-desacetoxy-17R,20S,24,25-tetrahydrofusid-21-amide (Compound 163),

21-N-(2′-{[2′-({2′-[(2′-aminoethyl)amino]ethyl}amino)ethyllaminoj ethyl)-3β-desacetoxy-17R,20S,24,25-tetrahydrofusid-21-amide (Compound 164),

21-N-[4′-({3′-[(4′-aminobutyl)amino]propyl}amino)butyl]-3β-desacetoxy-17R,20S,24,25-tetrahydrofusid-21-amide (Compound 165),

21-N-[3′-({4′-[(3′-aminopropyl)amino]butyl}amino)propyl]-3β-desacetoxy-17R,20S,24,25-tetrahydrofusid-21-amide (Compound 166),

21-N-{6′-aminohexyl}-3-OAc-17R,20S,24,25-tetrahydrofusid-21-amide (Compound 167),

21-N-{3′-[3′-aminoproyl)amino]propyl}-3-OAc-17R,20S,24,25-tetrahydrofusid-21-amide (Compound 168),

21-N-{3′-[(4′-aminobutyl)amino]propyl}-3-OAc-17R,20S,24,25-tetrahydrofusid-21-amide (Compound 169),

21-N-[2′-({3′-[(2′-aminoethyl)amino]propyl}amino)ethyl]-3-OAc-17R,20S,24,25-tetrahydrofusid-21-amide (Compound 170),

21-N-[3′-({2′-[(3′-aminopropyl)amino]ethyl}amino)propyl]-3-OAc-17R,20S,24,25-tetrahydrofusid-21-amide (Compound 171),

21-N-[3′-({3′-[(3′-aminopropyl)amino]propyl}amino)propyl]-3-OAc-17R,20S,24,25-tetrahydrofusid-21-amide (Compound 172),

21-N-[3′-({4′-[(3′-aminopropyl)amino]butyl}amino)propyl]-3-OAc-17R,20S,24,25-tetrahydrofusid-21-amide (Compound 173),

21-N-{3′-[3′-aminoproyl)amino]propyl}-3-OSO₃-11-desoxy-17,20,24,25-tetrahydrofusid-21-amide (Compound 174),

21-N-[2′-({3′-[(2′-aminoethyl)amino]propyl}amino)ethyl]-3-OSO₃-11-desoxy-17,20,24,25-tetrahydrofusid-21-amide (Compound 175),

21-N-{3′-[(4′-aminobutyl)amino]propyl}-3-OSO₃-11-desoxy-17,20,24,25-tetrahydrofusid-21-amide (Compound 176),

21-N-[3′-({4′-[(3′-aminopropyl)amino]butyl}amino)propyl]-3-OSO₃-11-desoxy-17,20,24,25-tetrahydrofusid-21-amide (Compound 177),

21-N-(2′-{[2′-({2′-[(2′-aminoethyl)amino]ethyl}amino)ethyl]amino}ethyl)-3-OSO₃-11-desoxy-17,20,24,25-tetrahydrofusid-21-amide (Compound 178),

21-N-[3′-({2′-[(3′-aminopropyl)amino]ethyl}amino)propyl]-3-OSO₃-11-desoxy-17,20,24,25-tetrahydrofusid-21-amide (Compound 179),

21-N-[4′-({3′-[(4′-aminobutyl)amino]propyl}amino)butyl]-3-OSO₃-11-desoxy-17,20,24,25-tetrahydrofusid-21-amide (Compound 180).

Naming of the above mentioned compounds is based on IUPAC for the branched polyamine side chain and on fusidane conventions for the steroid moiety. Naming has been assisted by using the program available at http://www2.acdlabs.com/ilab/

Pharmaceutical Compositions

Compositions of the invention comprise as an active component at least one compound of formula I or Ia (hereinafter referred to as the active ingredient) including acceptable salts and esters thereof, and optionally together with a pharmaceutically acceptable vehicle and/or diluent.

In said composition, the proportion of active ingredient to vehicle may vary from 0.5% to 100% by weight, in particular from about 0.1 to about 50% by weight. The compositions can be worked up to various pharmaceutical forms of presentation such as granulates, tablets, pills, dragees, suppositories, capsules, sustained-release tablets, suspensions, injection and may be filled in bottles or tubes or similar containers in accordance with accepted principles of pharmaceutical formulation, e.g. as disclosed in Remington: The Science and Practice of Pharmacy, 19^{th} Ed., Mack Publishing Company, 1995. Pharmaceutical organic or inorganic, solid or liquid carriers and/or diluents suitable for oral, enteral, parenteral or topical administration can be used to make up compositions containing the present compounds: Water, gelatine, lactose, starch, magnesium stearate, talc, vegetable and animal oils and fats, benzyl alcohol, gum, polyalkylene glycol, petroleum jelly, cocoa butter, lanolin, and other emulsifying agents, salts for varying the osmotic pressure or buffers for securing an appropriate pH-value of the composition can be used as auxiliary agents.

Furthermore, the composition may contain other therapeutically active components which can appropriately be administered together with the compounds of the invention in the treatment of infectious diseases such as other suitable antibiotics, in particular such antibiotics which may enhance the activity and/or prevent development of resistance. Such antibiotics include penicillins, cephalosporins, tetracyclines, rifamycins, erythromycins, lincomycin, clindamycin and fluoroquinolones. Other compounds which advantageously may be combined with the compounds of the invention, especially in topical preparations, include e.g. corticosteroids, such as hydrocortisone or triamcinolone. Alternatively, such other therapeutically active component(s) may be administered concomitantly (either simultaneously or sequentially) with the composition of the invention.

For granulates, tablets, capsules or dragees the pharmaceutical composition of the invention appropriately contains from 25% to 98% of the active ingredient of the invention, and in oral suspensions the corresponding amount is appropriately from 2% to 20% active ingredient.

When the active ingredient is administered in the form of salts with pharmaceutically acceptable non-toxic bases, preferred salts are for instance easily water-soluble or slightly soluble in water, in order to obtain a particular and appropriate rate of absorption.

As indicated above, the compounds of formula I and Ia and their salts may be included in pharmaceutical formulations, including suspensions, ointments and creams. A pharmaceutical preparation for oral administration may also be in form of a suspension of the active ingredient as such or in the form of a sparingly water-soluble pharmaceutically acceptable salt, the preparation containing from 20 to 100 mg per ml of vehicle. A pharmaceutical preparation for topical treatment may be in the form of an ointment or cream containing the active ingredient in an amount of from 0.5 to 50% of preparation. Topical preparations are favourable due to the stability towards sunlight and the relatively lipophilic nature of the present compounds.

The dose of the compounds of the invention may suitably be selected so that the desired activity may be achieved without serious adverse effects. In the human systemic therapy the compounds and their salts are conveniently administered (to adults) in dosage units containing no less than 50 mg and up to 1000 mg, preferably from 200 to 750 mg, calculated as the compound of formula I.

By the term “dosage unit” is meant a unitary, i.e. a single dose which is capable of being administered to a patient, and which may be readily handled and packed, remaining as a physically and chemically stable unit dose comprising either the active ingredient alone or in admixture with one or more solid or liquid pharmaceutical diluents or vehicles.

In the form of a dosage unit, the compound may be administered one or more times a day at appropriate intervals, always depending, however, on the condition of the patient, and in accordance with the prescription made by the medical practitioner.

Thus in systemic treatment a daily dosage will preferably be an amount of from 0.5 to 3 g of the active ingredient.

The term “usage unit” in connection with topical use means a unitary, i.e. a single dose capable of being administered topically to a patient in an application per square centimeter of the infected area of from 0.1 mg to 10 mg and preferably from 0.2 mg to 1 mg of the active ingredient in question.

If the composition is to be injected, a sealed ampoule, a vial or a similar container may be provided containing a parenterally acceptable sterile aqueous or oily injectable solution or dispersion of the active ingredient as the dosage unit.

The parenteral preparations are in particular useful in the treatment of conditions in which a quick response to the treatment is desirable. In the continuous therapy of patients suffering from infectious diseases, the tablets or capsules may be the appropriate form of pharmaceutical preparation owing to the prolonged effect obtained when the drug is given orally, in particular in the form of sustained-release tablets.

In the treatment of infectious diseases, such tablets may advantageously contain other active components as mentioned above.

In the method of treating patients suffering from infectious disease, the compound of formula I or Ia or an equivalent amount of a salt thereof may suitably be administered to patients in a dose of from 0.03 g to 0.7g/kg body weight per day in 1 to 3 doses, preferably from 0.5 g to 3 g per day. Preferably, the active ingredient is administered in the form of dosage units as indicated above.

Biological Activity

In vitro investigations have shown a relatively high potency of compounds of the invention against all bacteria including gram-positive and gram-negative strains (Staphylococci, Streptococci, Corynebacteriae, Mycobacteriae, Proteus, Propionibacterium, Pseudomonas, Neisseriae, E. coli) and fungal strains (Candida and Aspergillus). Biological tests have showed superior activity of some no compounds of the invention when compared with that reported for several natural squalamine analogues (WO 00/09137). The antibacterial activity of polyaminated fusidic acid analogues is also comparable to that of related compounds reported in the literature (Moore et al, 1993; Kikuchi et al., 1997; Rao et al., 2000) and to known broad spectrum antibiotics such as ampicillin (Kikuchi et al., 1997). In addition, the studies of post-antibiotic effects point towards a strong bactericidal effect of the compounds of the invention. Table 1 shows MIC (Minimum Inhibitory Concentration) values of compounds of the invention towards a number of bacterial and fungal strains. The potency of new polyaminated fusidic acid analogues is estimated by comparing the inhibition of growth of different microorganisms produced by known concentrations of the analogue to be examined and a reference compound such as fusidic acid. The microbiological assay set up is in agreement with the European Pharmacopoeia 3rd edition (1997). It is an agar diffusion method where the same volume of the tested solution is added to cavities in agar. The inhibition zones are function of the concentration of the fusidic acid analogue used. All assays are run with fusidic acid as reference substance.

TABLE 2
Selected compounds of the invention and their in vitro activities MIC (mg/l)

| Microorganism | | | | | | | | | | | | | | | Line- | Mupi- |
|---|---|---|---|---|---|---|---|---|---|---|---|---|---|---|---|---|
| strain | 121 | 123 | 125 | 131 | 133 | 145 | 146 | 152 | 154 | 172 | 173 | 177 | 180 | FA | zolid | rocin |
| S. aureus | 4 | 4 | 1 | 4 | 4 | 1 | 1 | 4 | 4 | 4 | 4 | 1 | 1 | 0.02 | 1 | 0.5 |
| CJ247 | | | | | | | | | | | | | | | | |
| S. aureus | 4 | 4 | 1 | 4 | 4 | 1 | 1 | 4 | 4 | 4 | 4 | 1 | 1 | 0.02 | 4 | 1 |
| CJ200 | | | | | | | | | | | | | | | | |
| S. aureus | 4 | 4 | 1 | 4 | 4 | 4 | 1 | 4 | 4 | 1 | 1 | 1 | 1 | 0.02 | 16 | 1 |
| CJ234R | | | | | | | | | | | | | | | | |
| S. aureus | 4 | 4 | 1 | 4 | 4 | 1 | 1 | 4 | 4 | 4 | 4 | 1 | 1 | 16 | 1 | 0.5 |
| CJ234F | | | | | | | | | | | | | | | | |
| S. aureus | 16 | 4 | 1 | 4 | 4 | 1 | 1 | 4 | 4 | 4 | 4 | 4 | 1 | 16 | — | — |
| N6 | | | | | | | | | | | | | | | | |
| S. epidermis | 4 | 4 | 1 | 4 | 4 | 1 | 1 | 2 | 1 | 4 | 1 | 1 | 4 | 0.02 | 0.25 | 0.04 |
| CK5 | | | | | | | | | | | | | | | | |
| Propionibacterium | 4 | 4 | 1 | 4 | 4 | 1 | 1 | — | — | 1 | 4 | 1 | 1 | 0.2 | 1 | — |
| acnes FN33 | | | | | | | | | | | | | | | | |
| Corynebacterium | 4 | 4 | 1 | 4 | 4 | 1 | 1 | 1 | 0.5 | 4 | 4 | 1 | 1 | 0.1 | — | — |
| xerosis FF | | | | | | | | | | | | | | | | |
| Streptococcus | 16 | 4 | 4 | 4 | 4 | 4 | 1 | — | — | 4 | 4 | 4 | 16 | 16 | 4 | 1 |
| pyogenes EC88 | | | | | | | | | | | | | | | | |
| Streptococcus | 16 | 4 | 16 | 4 | 4 | 4 | 1 | — | — | — | — | — | — | — | — | — |
| faecium EI19 | | | | | | | | | | | | | | | | |
| E. coli | 16 | 16 | 16 | 16 | 16 | 4 | 4 | 4 | 8 | 4 | 4 | 16 | 16 | >64 | — | — |
| HA165 | | | | | | | | | | | | | | | | |
| Pseudomonas | — | 16 | 16 | 4 | 16 | 4 | 16 | 8 | 16 | 4 | 4 | 16 | 64 | >64 | — | — |
| aeruginosa | | | | | | | | | | | | | | | | |
| BA17 | | | | | | | | | | | | | | | | |
| Saccaromyces | 16 | 16 | 16 | 16 | 32 | 4 | 4 | >125 | >125 | 64 | 4 | 125 | 125 | >64 | — | — |
| cervisiae ZZ7 | | | | | | | | | | | | | | | | |
| Candida albicans | 16 | 16 | 16 | 16 | 32 | >125 | >125 | >125 | >125 | 64 | 125 | 64 | 64 | >64 | — | — |
| ZA | | | | | | | | | | | | | | | | |
| Asbergillus niger | 64 | 4 | 16 | 16 | 32 | >125 | >125 | >125 | >125 | 64 | 125 | 16 | 4 | >64 | — | — |
| ZM35 | | | | | | | | | | | | | | | | |

Comment:
Very clear inhibition zones for all compounds listed in table 2 indicate bactericidal action.
FA = fusidic acid
— = missing MIC value
Strains:
FF = Corynebacterium xerosis
EC88 = Streptococcus pyrogenes
CJ234(F) = Staphylococcus aureus (MRSA#, Fus. resistant)
CJ(N6) = Staphylococcus aureus (Fus. resistant)
CJ247 = Staphylococcus aureus
CJ234(R) = Staphylococcus aureus (MRSA#, Rifampicin resistant)
CJ1200 = Staphylococcus aureus
CK5 = Staphylococcus epidermidis
#MRSA: meticilline resistant S. aureus
BA17 = Pseudomonas
HJ = Proteus
EI119(P) = Streptococcus faecium (Penicillin resistant)
ZA = Candida albicans
HA165 = E. coli
ZZ7 = Saccharomyces cerevisiae
FN33 = Propionibacterium
ZM6 = Aspergillus flavus
ZM35 = Aspergillus niger

The invention is further illustated in the following Preparations and Examples

### PREPARATIONS AND EXAMPLES

General

The following standard abbreviations are used throughout this disclosure:

Ac=acetyl

Et=ethyl

Ether=diethyl ether

DCC=dicyclohexylcarbodiimide

DMF=dimethylformamide

HPLC=high performance liquid chromatography

Me=methyl

THF=tetrahydrofuran

TLC=Thin Layer Chromatography

TMS=tetramethylsilyl

TEMPO=2,2,6,6-tetrametyl-1-piperidinyloxy free radical

NMR spectra were recorded at 300° K. on either a Bruker ARX300 or a Bruker DRX500 spectrometer equipped with a 5 mm qnp and a 5 mm broad band probe, respectively.

CD₃OD or CDCl₃ was used as solvent. All chemical shift are given in ppm 5 scala using tetramethylsilane (TMS δ=0.00 ppm) as internal reference.

Conventional ¹H, ¹³C and DEPT 135 spectra were obtained on most compounds.

Mass spectra were recorded on either a Micromass LC-QuattroII, or a high resolution Micromass AutoSpec sector instrument.

All reactions were conducted in pre-dried glass glassware under an atmosphere of argon and transfer of reagents were carried out using syringes. All solvents and reagents were of highest available quality and used as such with the exception of some liquid polyamine building blocks of moderate purity which were distilled prior to use. Reactions could be monitored by TLC analyses using 0.25 mm glass-coated silica plates (E. Merck 60 F254). Chromatography was performed on silica gel 60, 230-400 mesh (E. Merck) using mixtures of ethyl acetate and low boiling petroleum ether (succinimide esters)or mixtures of dichloromethan, methanol and aqueous ammonia as eluant (polyamines: compounds of the invention). Compounds were alternatively purified by reversed phase (RP-18) preparative HPLC using acetonitrile buffered with trifluoroacetic acid. All purified compounds were freeze-dried from water yielding white amorphous powder. Anhydrous solvents were prepared by storing analytical grade solvents over 4A molecular sieves a few days prior to use. The water content was measured before use on a Carl Fisher apparatus (typical water content: 5-12 ppm for chloroform and THF).

Preparations:

Fusidic Acid Type Starting Materials

The starting fusidic acid related analogues can be prepared according to various literature procedures starting from natural fusidanes like fusidic acid, helvolic acid, viridominic acids and compounds from the cephalosporin P family (see e.g. Godtfredsen and Vangedal, 1962; Arigoni et al., 1964; Godtfredsen et al., 1965ₐ and 1965_{b}; Godtfredsen et al., 1966; Diassi et al., 1966; von Daehne et al, 1979 and references cited therein) and by similar chemical modifications of the above-mentioned including hydrogenation of double bonds, dehydration reactions, sulphatation, acetylation, desacetylation and oxidations, well known to those skilled in the art.

Polyamine Building Blocks

Polyamine building blocks are generally chosen from those commercially available, e.g. those found in the Available Chemicals Directory (ACD) database, but can also be synthesised by methods known from the literature including such reactions as direct alkylation of amines, reductive amination and catalytic hydrogenation of amides to the corresponding amines (selected references describing various synthetic methods for the preparation of polyamine building blocks: Goodnow et al., 1990; Bergeron et al., 1994; Stromgaard et al., 1999; Gaell and Blagbrough, 2000; Kuksa et al., 2000 and references cited therein; Karigiannis and Papaioannou, 2000 and references cited therein).

General Methods:

Preparation of Compounds in which the Polyamine Building Block is Linked to C-21 of Fusidic Acid by an Amide Bond

Compounds of the invention where the polyamine building block is linked to the fusidane nucleus via an amide bond may be prepared from various steroids containing a carboxylic acid, e.g. from tetrahydrofusidic acid in scheme 1, and numerous polyamine building blocks as defined above. The carboxylic acid group of a fusidic acid derivative is first esterified to produce a reactive ester, for example a succinimide ester of by reacting the carboxylic acid group with N-hydroxysuccinimide in anhydrous THF in presence of dicyclocarbodiimide (Kikuchi et al., 1997). The succinimide ester may then be reacted with a polyamine building block by dissolving an excess of the polyamine in anhydrous chloroform under argon and then slowly adding a chloroform solution containing the activated ester (Kikuchi et al., 1997). The reactions are performed at room temperature and are completed between 6 and 24 hours. After this time the reaction mixture can be concentrated without additional aqueous work-up procedures and directly purified by reversed phase HPLC using mixtures of acetonitrile and water buffered with trifluoroacetic acid as eluent or column cromatography on silica gel using mixtures of dichloromethan, methanol and aqueous amonia as eluent. All compounds of the invention obtained using method A could be prepared using the reaction conditions described hereinafter for Compound 125.

Preparation of Compound 125 Using General Method A, Scheme 1

The method is illustrated by an example in Scheme 1 where the fusidic acid nucleus is represented by tetrahydrofusidic acid. Tetrahydrofusidic acid is first converted to the corresponding succinimide ester by reaction with N-hydroxysuccinimide in anhydrous THF in presence of dicyclocarbodiiunide. Spermine (3 equivalents) in anhydrous chloroform under argon is then slowly added over a period of 30 min to a chloroform solution containing tetrahydrofusidic acid N-succinimide ester. The reaction mixture is stirred at room temperature for 16 h after which time chloroform is evaporated under reduced pressure resulting a pale yellow oil. Pure Compound 125 is obtained after chromatography on silica gel using a T mixture of dichloromethan, methanol and 25% aqueaous ammonia as eluent. A white powder of pure Compound 125 is obtained after freeze drying of purified product in yields ranging from 60-90%.

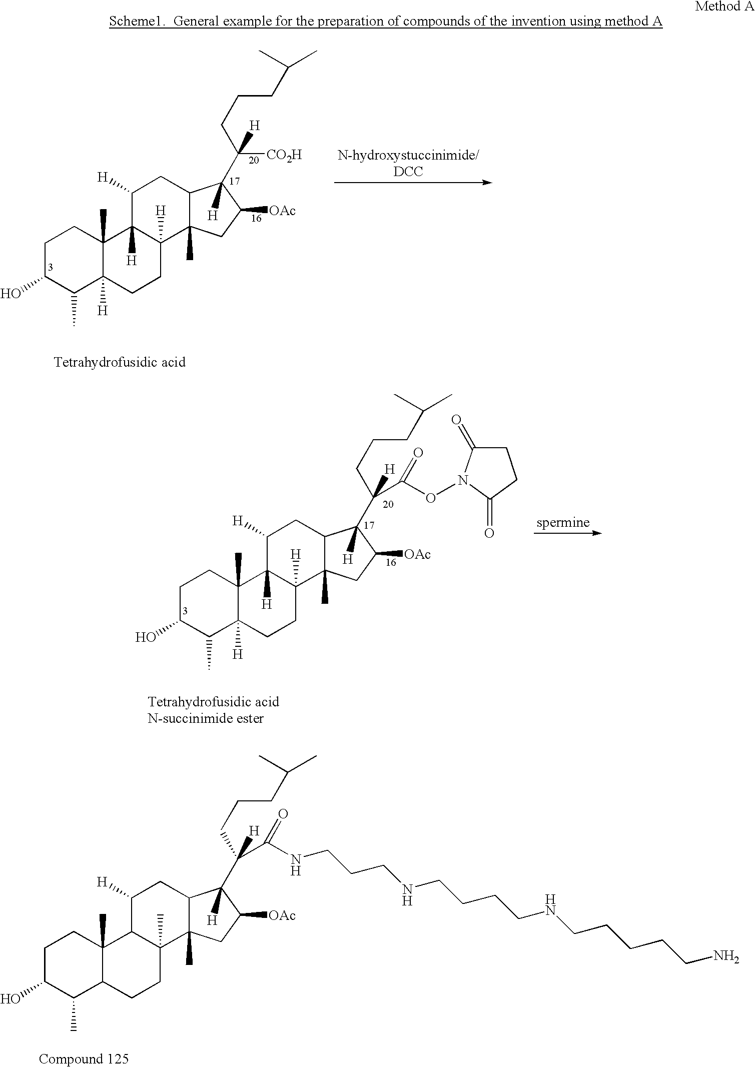

Alternative Preparation of Compounds in which the Polyamine is Linked to C-21 of Fusidic Acid by an Amide Bond

Alternatively, the compounds of the invention can be prepared by reacting anhydrides of fusidic acid derivatives, e.g. fusidic acid anhydride in scheme 2, with excess of the branched polyamine building blocks (Scheme 2).

Preparation of Compound 107 using General Method B, Scheme 2

The method is illustrated by an example in Scheme 2 where the fusidic acid nucleus is represented by natural fusidic acid. Fusidic acid (1 eauivalent) is dissolved in anhydrous DMF and and N,N-dicyclohexylcarbodiimide (2.2 equivalents) was added. The resulting reaction mixture was heated at 50° C. for 24 hours. The reaction mixture was diluted with ethyl acetate, washed with saturated aqueous sodium bicarbonate and brine, dried over anhydrous sodium sulphate and concentrated under reduced pressure. Pure anhydride is obtained either by crystallisation from hot methanol or by chromatography on silica gel using mixtures of ethyl acetate and petroleum ether as eluant. Spermine (3 equivalents) in anhydrous chloroform under argon is then slowly added over a period of 30 min to a chloroform solution containing fusidic acid anhydride. The reaction mixture is stirred at room temperature for 16 h after which time chloroform is evaporated under reduced pressure resulting a pale yellow oil. Pure Compound 107 is obtained after chromatography on silica gel using a mixture of dichloromethan, methanol and 25% aqueaous ammonia as eluant. A white powder of pure Compound 107 is obtained after freeze drying of purified product in yields ranging from 70-90%.

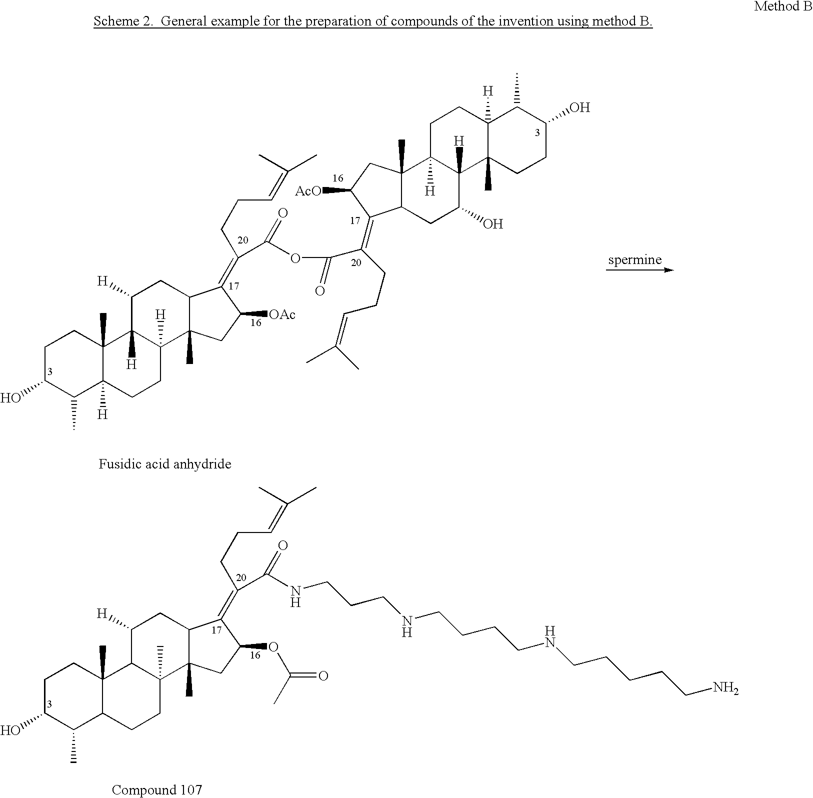

Reduction of the Amide Bond

The amide bonding resulting from the reaction of a polyamine and a succinimide ester or carboxylic acid anhydride described in scheme 1 and 2 respectively (e.g. compounds of Compound 125 and 107) can be reduced to the corresponding amine by reacting the amide with a 10 fold excess of diborane in refluxing THF for 5-10 hours, as depicted in scheme 3. The reaction mixture is subsequently acidified with 4N aqueous hydrochloric acid to pH 1 and stirred vigorously for 24 hours. The reaction mixture is then freeze dried and the resulting white powder is purified on silica gel using a mixture of dichloromethan, methanol and 25% aqueaous ammonia as eluant. A white powder is obtained after freeze drying of purified product.

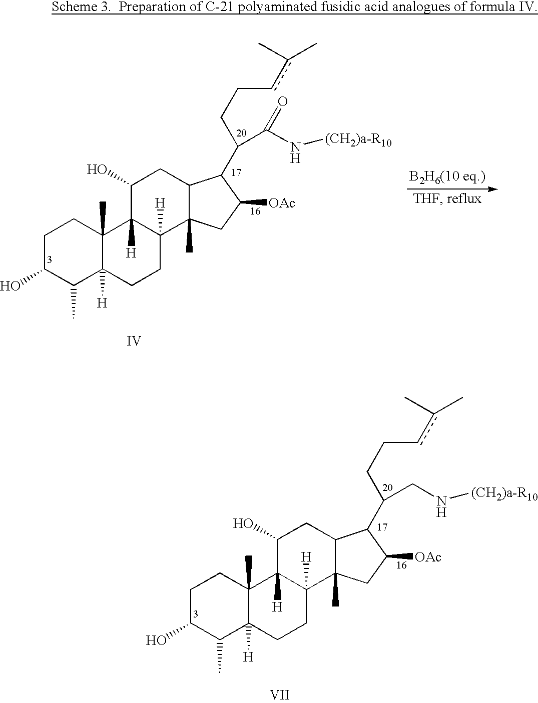

Introduction of Polyamines by Reductive Amination of Ketones

Compounds of the invention where the polyamine moiety is linked to various sites of the fusidic acid nucleus can be prepared from steroid analogues containing a keto or aldehyde functionality where substitution with the branched polyamine is desired. The appropriate fusidic acid having an aldehyde or a keto functionality can be obtained from various known derivatives of fusidic acid by methods known to those skilled in the art (e.g. oxidation method of hydroxy groups, allylic oxidation and partial reduction of carboxylic esters, etc.). For example 3- or 16-keto derivatives of fusidic acid can be reacted directly with the unprotected polyamine building block by means of reductive amination using methods reported for the preparation of synthetic squalamines (Pechulis et al., 1995; Weis et al., 1999; Kinney et al., 2000).

Preparation of Compound 113 using General Method C, Scheme 4

The method is illustrated by an example in Scheme 4 where the fusidic acid nucleus is represented by 3-keto fusidic acid. Fusidic acid is first oxidised to 3-keto fusidic acid by means of CrO₃, altenatively by pyridinium dichromate, Dess-Martin periodinane or by a Swem protocol. To a solution of 3-keto fusidic acid (1 equivalent) in methanol was added successively N,N′-bis(3-aminopropyl)-1,3-propanediamine (3 equivalents), acetic acid and NaBH(OAc)₃ (3 equivalents) and the resultin reaction mixture was stirred for 6-16 h, after which time methanol is evaporated under reduced pressure providing a pale yellow oil. Pure Compound 113 is obtained after chromatography on silica gel using a mixture of dichloromethan, methanol and 25% aqueaous ammonia as eluant. A white powder of pure Compound 113 is obtained after freeze drying of purified product in yields ranging from 70-85%.

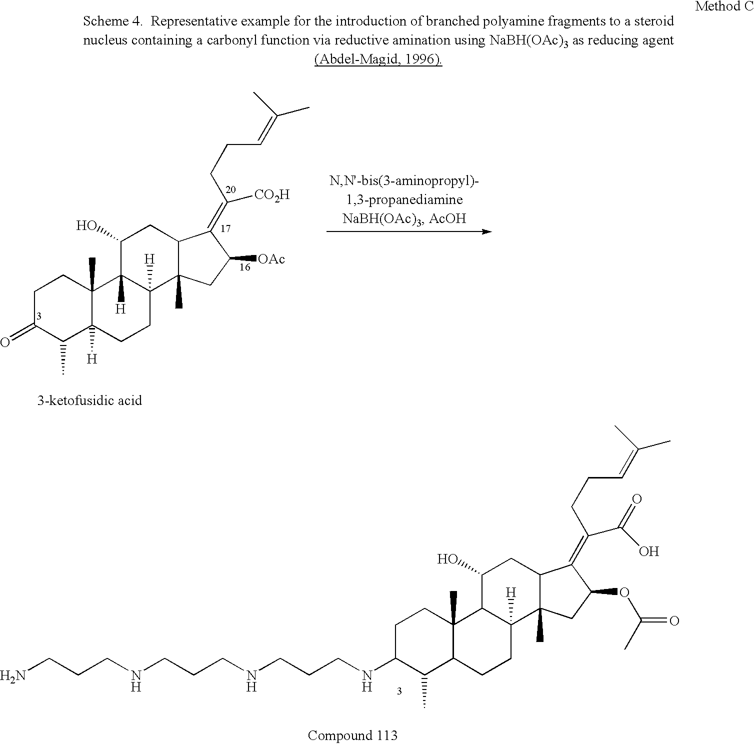

Sulfation of Free Hydroxy Groups:

All compounds of the invention containing one or several free hydroxy groups can optionally be sulfated either selectively at one hydroxy group or at several hydroxy group using stoichiometric or excess amounts of sulfur trioxide-pyridine complex, respectively as reported in the litterature (Kinney et al., 2000). Sulfatation is carried out prior to coupling reactions A, B and C.

Acetylation of Hydroxy Groups

Acetylation of the free hydroxy groups of fusidic acid derivatives is carried out using an excess of acetic acid anhydride in pyridine at room temperature under anhydrous conditions.

Reduction of Double Bonds

Double bonds of fusidic acid derivatives are carried out by means of catalytic hydrogenation using palladium on carbon as catalyst and acetic acid as solvent. The reactions are shaken for 6-20 h at room temperature.

Dehydration of 11-OH

Dehydration of 11-OH of fusidic acid derivatives is achieved by trating fusidic acid derivatives by excess thionyl chloride in pyridine and dichloromethan at 0° C. under anhydrous conditions.

Removal of the 16-Acetoxy Group

The 16-acetoxy group of fusidic acid derivatives can be removed by reacting the corresponding methyl ester in refluxing anhydrous methanol in presence of excess magnesium turnings under anhydrous conditions. The methyl ester is then removed by refluxing in aqueous sodium hydroxide for 1 h.

Purification of the Compounds of the Invention:

The resulting compounds of the invention can be purified by column chromatography on silca gel 60 (E. Merck), 230400 mesh using mixtures of dichloromethan, methanol and aqueous ammonia as eluant. Alternatively, the compounds of the invention can be purified by reversed phase preparative high performance liquid chromatography (HPLC) using acetonitrile buffered with trifluoroacetic acid or acetic acid as eluant.

Oxidation of Hydroxy Groups

Keto derivatives can be obtained by oxidation of the corresponding hydroxy group of a fusidic acid derivative by various methods such as CrO₃ in DMF or dichloromethan, pyridinium dichromate, pyridinium chlorochromateDess-Martin periodinane by a Swern protocol or by using radical reagent such as TEMPO.

Preparation of N-succinimide Esters of Fusidic Acid Analogues, General Method D:

The fusidic acid derivative (1 equivalent) was dissolved in anhydrous THF. To the solution was added successively N-hydroxysuccinimide (1.1 eauivalent) and N,N-dicyclohexylcarbodiimide (1.2 equivalent). The resulting reaction mixture was stirred at room temperature for 20 hours. The reaction was filtered, the filtrate was concentrated under reduced pressure and redissolved in ethyl acetate. The organic solution was washed with saturated aqueous sodium bicarbonate and brine, dried over anhydrous sodium sulfate and concentrated under reduced pressure. Pure N-succinimide esters were obtained either by crystallisation from hot methanol or by chromatography on silica gel using mixtures of ethyl acetate and petroleum ether as eluant.

Preparation of Anhydrides of Fusidic Acid Analogues, General Method E:

The fusidic acid derivative was dissolved in anhydrous DMF and and N,N-dicyclohexylcarbodiimide (2.2 equivalents) was added. The resulting reaction mixture was heated at 50° C. for 24 hours. The reaction mixture was diluted with ethyl acetate, washed with saturated aqueous sodium bicarbonate and brine, dried over anhydrous sodium sulphate and concentrated under reduced pressure. Pure anhydride was obtained either by crystallisation from hot methanol or by chromatography on silica gel using mixtures of ethyl acetate and petroleum ether as eluant.

Preparation 1: Fusidic Anhydride, Compound 1

The title compound was prepared from fusidic acid according to general method E.

¹³C NMR (CDCl₃): 171.0, 164.7, 153.2, 132.6, 129.1, 123.0, 74.2, 71.4, 68.3, 49.2, 48.8, 44.7, 39.5, 39.1, 37.0, 36.2, 36.1, 35.5, 32.3, 30.3, 30.0, 29.0, 28.9, 25.8, 24.2, 22.8, 21.1, 20.8, 18.0, 17.9, 15.9.

Preparation 2: 3β-Hydroxy-17R,20S,24,25-Tetrahydrofusidic Acid Succinimide Ester, Compound 2

The title compound was prepared from 3β-hydroxy-17R,20S,24,25-tetrahydrofiusidic acid according to method D.

¹³C NMR (CDCl₃): 171.1, 170.6, 169.8, 168.7, 79.3, 67.9, 49.7, 49.6, 49.1, 46.9, 42.7, 40.4, 40.1, 40.0, 39.9, 38.7, 36.6, 35.4, 34.1, 32.6, 32.2, 31.8, 27.9, 25.8, 25.5, 25.0, 24.6, 23.9, 23.3, 22.5, 21.3, 21.3, 16.7, 15.4.

Preparation 3: 17R,20S,24,25-Tetrahydrofusidic Acid Succinimide Ester, Compound 3

The title compound was prepared from 17R,20S,24,25-tetrahydrofusidic acid according to method D.

¹³C NMR (CDCl₃): 171.1, 170.6, 169.8, 168.8, 79.0, 71.6, 67.9, 49.8, 49.5, 49.0, 46.6, 40.3, 40.2, 40.0, 38.7, 37.0, 36.5, 36.3, 35.2, 32.4, 31.9, 30.1, 30.0, 28.0, 25.7, 24.6, 23.4, 22.9, 22.5, 22.5, 21.3, 21.1, by 16.8, 16.0.

Preparation 4: 17S,20R-Dihydrofusidic Acid Succinimide Ester, Compound 4

The title compound was prepared from 17S,20R-dihydrofusidic acid according to method D.

¹³C NMR (CDCl₃): 171.3, 170.1, 169.2, 132.5, 123.1, 114.0, 75.9, 71.5, 68.6, 49.3, 44.1, 43.4, 41.2, 40.6, 39.1, 37.2, 36.5, 36.1, 34.5, 34.0, 33.3, 32.7, 30.2, 30.1, 25.7, 25.6, 25.3, 23.9, 22.4, 21.4, 20.9, 18.3, 17.7, 16.0.

Preparation 5: 11-Desoxy-17R,20S,24,25-Tetrahydrofusidic Acid Succinimide Ester, Compound 5

The title compound was prepared from 11-desoxy-17R,20S,24,25-tetrahydrofusidic acid according to method D.

¹³C NMR (CDCl₃):170.8, 170.6, 168.9, 77.4, 71.8, 49.2, 49.0, 45.0, 44.5, 44.2, 39.9, 39.4, 38.7, 37.8, 36.4, 34.9, 33.4, 30.6, 30.0, 28.6,27.9, 25.6, 25.1, 25.0, 22.9, 22.6, 22.5, 21.3, 20.2, 20.2, 17.4, 16.0.

Preparation 6: 17R,20R,S,24,25-Tetrahydro-16-Desoxyfusidic Acid Succinimide Ester, Compound 6

The title compounds was prepared from two separate C-20 epimers of 17R,20,24,25-tetrahydro-16-desoxyfusidic acid according to method D.

¹³C NMR (CDCl₃):

C-20 epimer-1: 170.6, 169.9, 168.9, 71.6, 68.5, 51.0, 49.9, 43.8, 42.7, 40.2, 38.7, 37.0, 36.9, 36.1, 34.4, 32.4, 32.0, 30.5, 30.1, 29.9, 27.8, 25.6, 25.4, 23.3, 22.7, 22.6, 22.5, 21.4, 16.0, 15.6 C-20 epimer-2: 172.1, 170.0, 168.9, 71.7, 68.1, 51.0, 50.1, 48.8, 42.6, 40.9, 40.3, 38.8, 37.1, 36.4, 35.7, 32.9, 32.5, 30.8, 30.2, 30.0, 28.2, 27.9, 25.7, 25.6, 24.8, 23.4, 22.8, 22.6, 22.5, 21.1, 16.1.

Preparation 7: 13(17)-en-17R,20,24,25-Tetrahydrofusidic Acid Succinimide Ester, Compound 7

The title compound was prepared from 13(17)-en-17R,20,24,25-tetrahydrofusidic acid according to method 2.

¹³C NMR (CDCl₃): 169.1, 168.7, 143.2, 130.5, 71.5, 69.9, 57.7, 55.8, 51.4, 41.6, 41.3, 38.4, 37.7, 36.8, 35.1, 34.2, 30.6, 30.4, 29.8, 29.8, 29.4, 29.3, 27.8, 25.6, 24.6, 24.2, 23.2, 22.7, 22.6, 22.5, 21.7, 15.9.

Preparation 8: 16(17)-en-17R,20R,S,24,25-Tetrahydrofusidic Acid Succinimide Ester, Compound 8

The title compound was prepared from 16(17)-en-17R,20,24,25-tetrahydrofusidic acid according to method D.

NMR (CDCl₃) (1:1 mixture of C-20 epimers): 169.3, 169.2, 169.1, 140.3, 140.2, 128.4, 127.1, 77.2, 71.7, 71.6, 68.7, 68.6, 54.1, 53.8, 50.6, 50.5, 49.2, 44.1, 43.5, 43.1, 42.7, 40.1, 40.1, 39.0, 38.9, 38.6, 38.6, 37.4, 36.6, 36.6, 36.2, 34.0, 32.7, 32.6, 32.5, 31.9, 30.6, 30.4, 30.3, 30.2, 30.2, 30.2, 27.8, 25.6, 25.3, 25.2, 25.1, 25.0, 25.0, 22.6, 22.5, 20.9, 17.6, 17.6, 16.1.

### EXAMPLES OF COMPOUNDS OF THE INVENTIONS PREPARED BY METHOD A, B OR C

| Ex- | | | | |
|---|---|---|---|---|
| ample | | | | |
| no./ | | | | |
| Comp. | | Steroid starting | Polyamine starting | |
| no. | Method | material | material | Structure of compound |
| 1/101 | B | Fusidic acid anhydride | 1,3-diamino-propane | 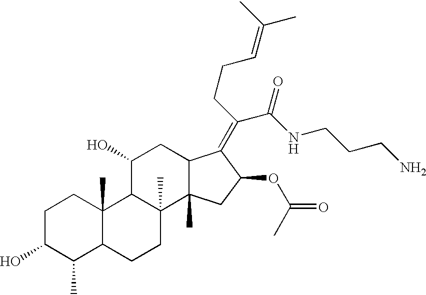 |

¹³C NMR (CD₃OD), δ/ppm: 174.5, 172.4, 143.4, 135.8, 133.2, 124.5, 75.2, 72.4, 68.6, 50.7, 44.6, 40.7,
40.3, 39.9, 38.2, 37.9, 37.9, 37.4, 36.9, 32.9, 31.1, 31.0, 30.5, 28.8, 25.9, 23.9, 23.8, 22.4, 21.2, 17.9, 17.9,
16.5

| | | | | |
|---|---|---|---|---|
| 2/102 | B | Fusidic acid anhydride | diethylenetriamine | 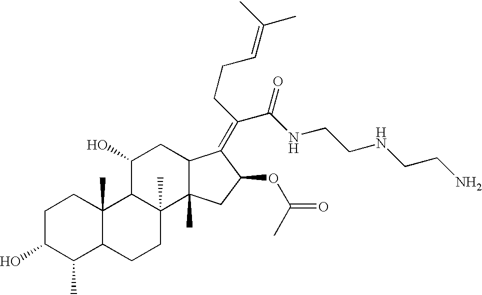 |

NMR (CD₃OD), δ/ppm: 174.6, 172.4, 143.4, 135.7, 133.2, 124.5, 75.2, 72.4, 68.6, 51.4, 50.7, 44.6, 41.5,
40.7, 40.3, 40.2, 38.2, 37.8, 37.4, 36.8, 32.9, 31.0, 31.0, 30.5, 28.8, 25.9, 23.8, 22.4, 21.2, 17.9, 17.9, 16.5

| | | | | |
|---|---|---|---|---|
| 3/103 | B | Fusidic acid anhydride | 3,3′-diamino-dipropyl- amine | 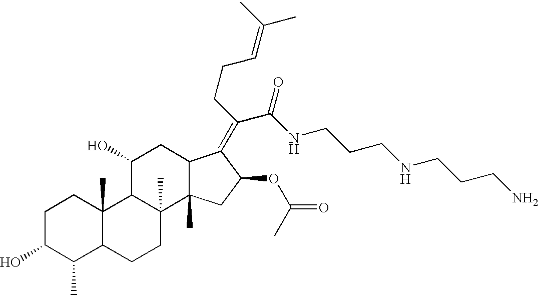 |

¹³C NMR (CD₃OD), δ/ppm: 171.2, 171.1, 140.8, 135.9, 132.2, 123.5, 73.4, 71.4, 68.2, 49.5, 49.2, 48.4,
47.8, 43.2, 40.6, 39.6, 39.5, 39.3, 37.0, 36.4, 36.0, 35.6, 34.1, 32.3, 30.2, 30.0, 29.2, 28.0, 27.8, 25.8, 23.9,
23.0, 21.1, 20.9, 17.9, 17.6, 16.0

| | | | | |
|---|---|---|---|---|
| 4/104 | B | Fusidic acid anhydride | spermidine | 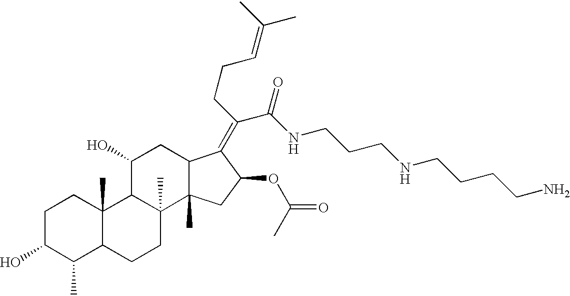 |

¹³C NMR (CD₃OD), δ/ppm: 174.5, 172.4, 143.5, 135.7, 133.2, 124.5, 75.2, 72.4, 68.6, 50.7, 50.3, 50.1,
48.3, 47.9, 44.6, 42.0, 40.7, 40.3, 38.5, 38.2, 37.9, 37.4, 36.9, 32.9, 31.1, 31.0, 30.5,
30.5, 28.9, 27.8, 25.9, 23.9, 23.8, 22.4, 21.2, 17.9, 16.5

| | | | | |
|---|---|---|---|---|
| 5/105 | B | Fusidic acid anhydride | N,N′-bis(2-amino- ethyl)-1,3-propane- diamine | 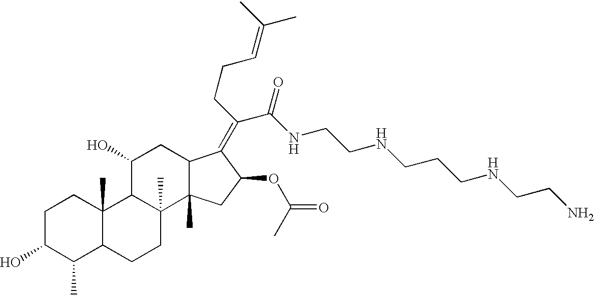 |

¹³C NMR (CD₃OD), d/ppm: 174.6, 172.4, 143.5, 135.7, 133.3, 124.5, 75.2, 72.5, 68.6, 52.4, 50.7, 48.6,
44.6, 41.6, 40.7, 40.3, 40.1, 38.2, 37.9, 37.4, 36.9, 32.9, 31.0, 30.5, 30.4, 28.8, 25.9, 23.9, 23.8, 22.4, 21.2,
18.0, 17.9, 16.5

| | | | | |
|---|---|---|---|---|
| 6/106 | B | Fusidic acid anhydride | N,N′-bis(3-amino- propyl)-1,3-propane- diamine | 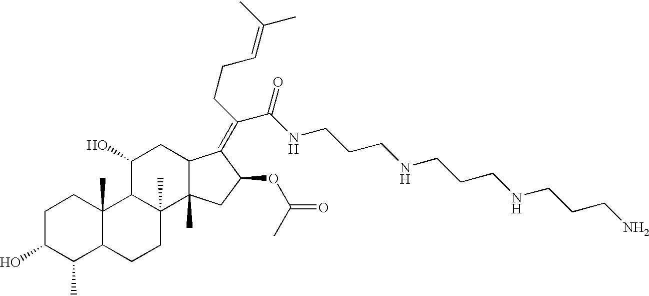 |

¹³C NMR(CD₃OD), δ/ppm: 171.1, 140.7, 136.0, 132.1, 123.6, 73.5, 71.4, 68.0, 50.6, 49.4, 49.2, 48.5,
48.4, 48.3, 48.0, 43.2, 40.4, 39.8, 39.5, 39.3, 36.9, 36.5, 36.0, 35.8, 33.3, 32.2, 30.7, 30.1, 30.0, 29.3, 28.1,
27.8, 25.8, 23.9, 23.0, 21.1, 20.9, 17.9, 17.6, 16.0

| | | | | |
|---|---|---|---|---|
| 7/107 | B | Fusidic acid anhydride | N,N′-bis(3-amino- propyl)-ethylene- diamine | 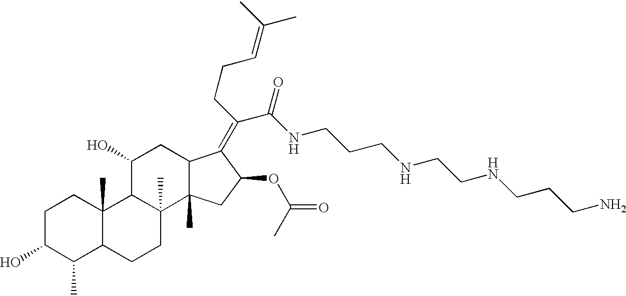 |

¹³C NMR (CD₃OD), δ/ppm: 174.4, 172.4, 143.4, 135.8, 133.2, 124.5, 75.2, 72.4, 68.6, 50.7, 50.5, 48.0,
44.6, 40.7, 40.6, 40.3, 38.5, 38.2, 37.9, 37.4, 36.9, 32.9, 32.8, 31.1, 31.0, 30.5, 29.9, 28.9, 28.3, 2.8.2, 25.9,
23.9, 23.8, 22.4, 21.1, 18.0, 17.9, 16.5
MS (direct inlet ) m/z = 701.55 (M + H)⁺, 321.48, 203.21, 89.06

| | | | | |
|---|---|---|---|---|
| 8/108 | B | Fusidic acid anhydride | spermine | 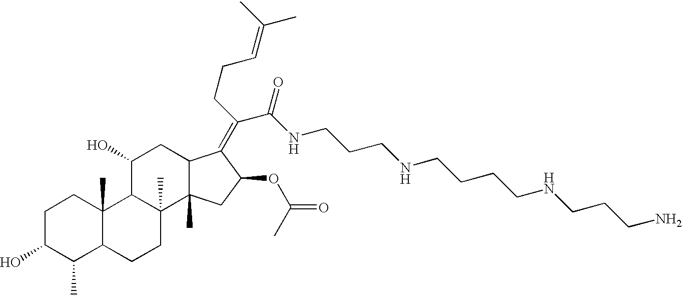 |

¹³C NMR (CD₃OD), δ/ppm: 178.7, 72.5, 69.3, 54.1, 51.8, 51.7, 48.1, 43.8, 42.4, 41.5, 40.6, 40.2, 38.3,
38.2, 38.0, 37.1, 36.8, 33.1, 32.7, 32.6, 31.7, 31.2, 31.1, 30.1, 29.2, 28.6, 26.6, 23.8, 23.2, 22.9, 22.7, 16.6,
16.4

| | | | | |
|---|---|---|---|---|
| 9/109 | B | Fusidic acid anhydride | N,N′-bis(4-amino- butyl)-1,3-propane- diamine | 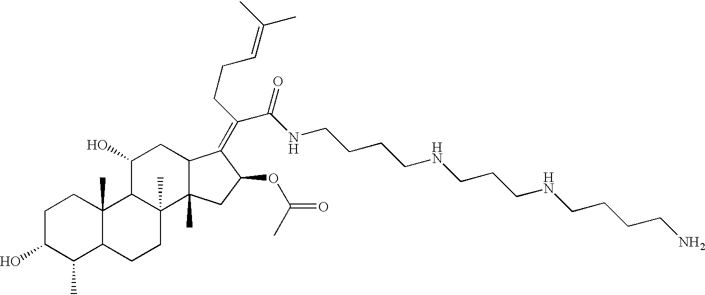 |

¹³C NMR( (CD₃OD), δ/ppm: 174.3, 172.4, 143.2, 135.9, 133.2, 124.6, 75.3, 72.4, 68.6, 50.7, 50.1, 48.6,
44.5, 41.9, 40.7, 40.4, 40.3, 38.2, 37.9, 37.4, 36.9, 32.9, 31.1, 31.0, 30.5, 30.4, 29.5, 28.8, 28.0, 27.8, 27.6,
25.9, 23.9, 23.8, 22.4, 21.2, 18.0, 17.9, 16.5

| | | | | |
|---|---|---|---|---|
| 10/110 | B | Fusidic acid anhydride | 6,6′-diamino- dihexylamine | 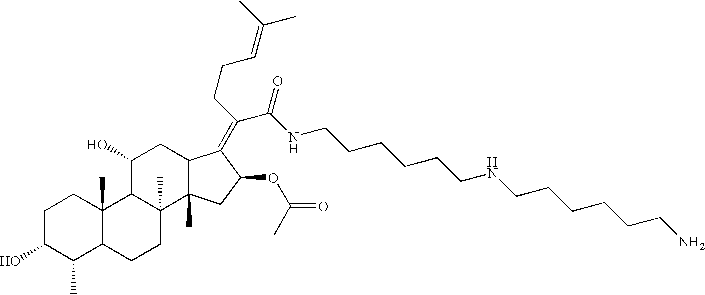 |

¹³C NMR (CD₃OD), δ/ppm: 174.3, 172.4, 143.1, 135.8, 133.2, 124.6, 75.3, 72.5, 68.6, 50.7, 50.6, 50.6,
44.5, 42.4, 40.7, 40.6, 40.3, 38.2, 37.9, 37.4, 36.9, 33.4, 32.9, 31.1, 30.5, 30.3, 30.2, 28.8, 28.3, 28.1, 28.1,
27.9, 25.9, 23.9, 22.4, 21.2, 18.0, 17.9, 16.5

| | | | | |
|---|---|---|---|---|
| 11/111 | A | Fusidic acid anhydride | 8,8′-diamino-dioctyl- amine | 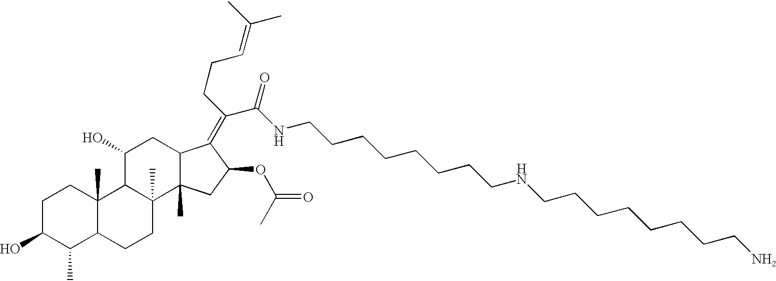 |

¹³C NMR (CD₃OD), δ/ppm: 174.3, 172.4, 143.1, 135.8, 133.1, 124.6, 75.3, 72.5, 68.6, 50.7, 44.5, 42.5,
40.7, 40.6, 40.3, 38.2, 37.9, 37.4, 36.9, 33.6, 32.9, 31.1, 30.6, 30.6, 30.4, 30.3, 28.8, 28.4, 28.4, 28.1, 28.0,
25.9, 23.8, 22.4, 21.2, 18.0, 17.9, 16.5

| | | | | |
|---|---|---|---|---|
| 12/112 | B | Fusidic acid anhydride | tetra- ethylene-pentamine | 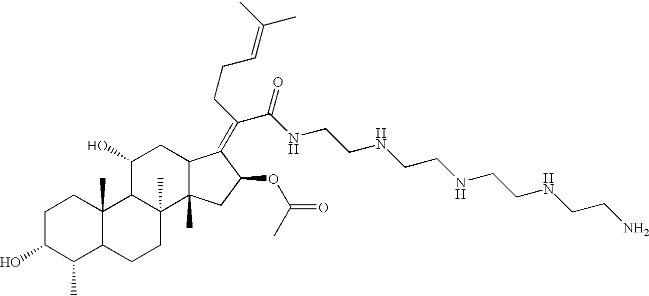 |

¹³C NMR (CD₃OD), δ/ppm: 171.5, 171.1, 141.0, 135.5, 132.1, 123.6, 73.9, 71.3, 68.1, 51.3, 49.4, 48.9,
48.6, 48.2, 43.2, 41.2, 39.5, 39.3, 38.9, 36.9, 36.5, 36.0, 35.7, 32.2, 30.2, 30.0, 29.5, 28.1, 25.8, 24.0, 23.1,
21.1, 20.9, 17.9, 17.7, 16.0

| | | | | |
|---|---|---|---|---|
| 13/113 | C | Fusidic acid anhydride | N,N′-bis(3-amino- propyl)-1,3-propane- diamine | 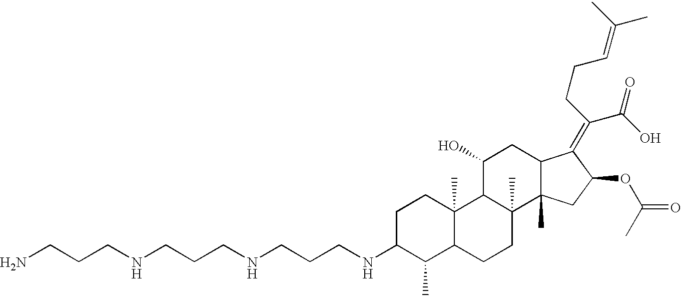 |

¹³C NMR (CD₃OD), δ/ppm: 179.5, 173.4, 139.9, 137.5, 132.3, 125.6, 75.9, 68.9, 61.1, 50.8, 43.6, 40.8,
40.3, 40.2, 37.7, 37.6, 37.3, 31.9, 31.1, 30.9, 30.7, 29.3, 28.9, 27.5, 25.9, 25.4, 24.6, 23.7, 22.6, 21,2, 18.0,
17.7, 16.7

| | | | | |
|---|---|---|---|---|
| 14/114 | C | Fusidic acid anhydride | N,N′-bis(2-amino- ethyl)-1,3-propane- diamine | 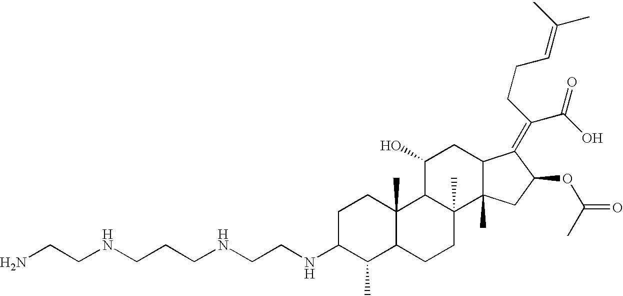 |

¹³C NMR (CD₃OD), δ/ppm: 180.3, 179.5, 173.2, 139.4, 138.3, 132.4, 125.5, 75.9, 68.9, 60.4, 50.8, 50.0,
49.9, 48.1, 47.9, 47.6, 45.5, 43.7, 40.8, 40.3, 40.1, 37.7, 37.5, 37.4, 31.9, 31.0, 29.3, 27.9, 26.0, 24.6, 24.2,
23.5, 22.7, 21.3, 18.0, 17.7, 16.7

| | | | | |
|---|---|---|---|---|
| 15/115 | C | Fusidic acid anhydride | N,N′-bis(3-amino- propyl)-ethylene- diamine | 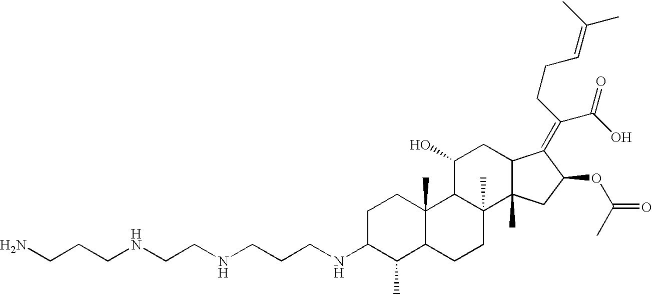 |

¹³C NMR (CD₃OD), δ/ppm: 179.6, 173.3, 139.7, 137.6, 132.3, 125.6, 75.9, 68.9, 61.0, 50.8, 50.0, 49.9,
48.6, 43.6, 40.8, 40.3, 40.1, 37.6, 37.6, 37.4, 37.2, 31.8, 31.1, 30.5, 30.5, 29.2, 27.6, 25.9, 25.1, 24.7, 23.5,
22.7, 21.2, 18.0, 17.6, 16.6

| | | | | |
|---|---|---|---|---|
| 16/116 | A | Tetrahydrofusidic acid-N-succinimide ester | tetra- ethylene-tetramine | 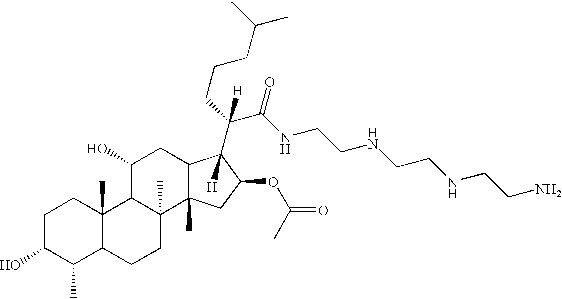 |

¹³C NMR (CD₃OD), δ/ppm: 177.8, 172.6, 80.5, 72.5, 68.9, 51.9, 51.3, 50.4, 50.1, 41.7, 41.4, 41.1, 40.1,
40.1, 38.3, 38.0, 37.0, 36.5, 33.0, 32.0, 31.1, 29.1, 26.3, 23.9, 23.4, 23.1, 22.9, 22.6, 21.4, 17.1, 16.5

| | | | | |
|---|---|---|---|---|
| 17/117 | A | Tetrahydrofusidic acid-N-succinimide ester | diethylenetriamine | 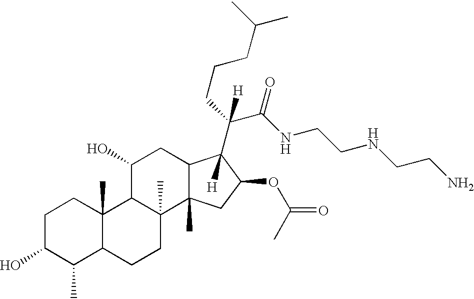 |

¹³C NMR (CD₃OD), δ/ppm: 177.8, 172.6, 80.5, 72.5, 68.9, 51.9, 51.5, 51.3, 50.4, 50.1, 41.7, 41.5, 41.4,
41.1, 40.2, 40.1, 38.3, 38.0, 37.1, 36.5, 33.1, 32.0, 31.1, 29.1, 26.3, 23.9, 23.4, 23.1, 22.9, 22.6, 21.4, 17.1,
16.5

| | | | | |
|---|---|---|---|---|
| 18/118 | C | Tetrahydrofusidic acid-N-succinimide ester | 1,6-hexanediamine | 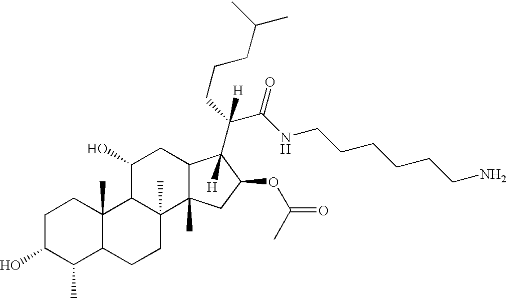 |

¹³C NMR (CD₃OD), δ/ppm: 177.4, 172.6, 80.1, 72.5, 69.0, 51.4, 51.3, 50.3, 50.1, 42.3, 41.6, 41.4, 41.1,
40.3, 40.1, 38.3, 37.9, 37.0, 36.3, 33.2, 33.1, 31.7, 31.0, 30.3, 29.2, 28.0, 27.7, 26.4, 23.9, 23.3, 23.1, 23.0,
22.6, 21.5, 17.1, 16.5

| | | | | |
|---|---|---|---|---|
| 19/119 | A | Tetrahydrofusidic acid-N-succinimide ester | 1,3-diamino-propane | 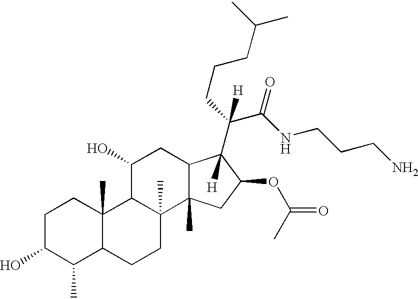 |

NMR (CD₃OD), δ/ppm: 177.7, 172.6, 80.2, 72.5, 68.9, 51.5, 51.3, 50.3, 50.1, 41.6, 41.4, 41.1, 40.1, 39.9,
38.3, 38.0, 37.7, 37.1, 36.4, 33.1, 32.8, 31.7, 31.1, 29.1, 26.4, 23.9, 23.3, 23.1, 22.9, 22.6, 21.4, 17.1, 16.5

| | | | | |
|---|---|---|---|---|
| 20/120 | A | Tetrahydrofusidic acid-N-succinimide ester | 3,3′-diamino-di- propylamine | 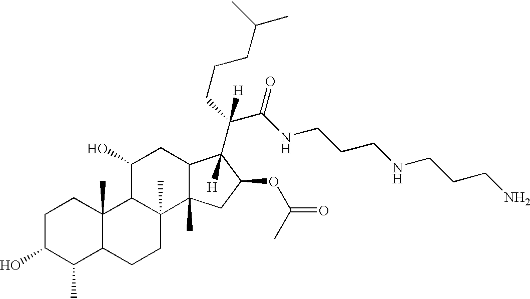 |

¹³C NMR (CD₃OD), δ/ppm: 177.5, 172.6, 80.2, 72.5, 68.9, 51.6, 51.3, 50.4, 50.1, 48.4, 41.6, 41.4, 41.1,
40.6, 40.1, 38.3, 38.0, 37.1, 36.4, 33.1, 32.6, 31.8, 31.1, 30.1, 29.2, 26.4, 23.9, 23.3, 23.2, 23.0, 22.6, 21.4,
17.1, 16.5

| | | | | |
|---|---|---|---|---|
| 21/121 | A | Tetrahydrofusidic acid-N-succinimide ester | spermidine | 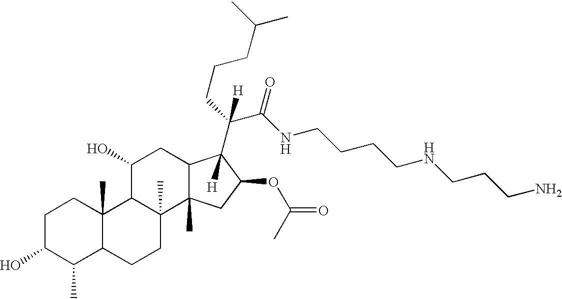 |

¹³C NMR (CD₃OD), δ/ppm: 177.7, 172.6, 80.2, 72.5, 68.9, 51.5, 51.3, 50.3, 50.2, 50.1, 47.8, 41.5, 41.4,
41.1, 40.1, 38.3, 38.1, 38.0, 37.1, 36.4, 33.1, 31.8, 31.1, 29.8, 29.4, 29.2, 27.5, 26.4, 23.9, 23.3, 23.2, 23.0,
22.6, 21.4, 17.1, 16.5

| | | | | |
|---|---|---|---|---|
| 22/122 | A | Tetrahydrofusidic acid-N-succinimide ester | N,N′-bis(2-amino- ethyl)-1,3-propane- diamine | 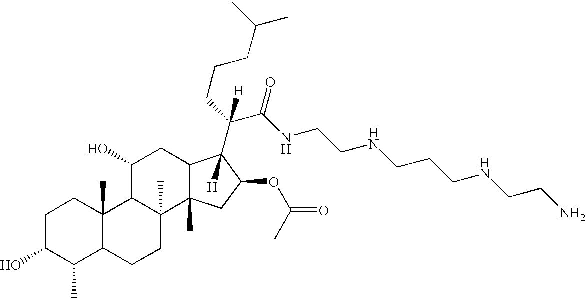 |

¹³C NMR (CD₃OD), δ/ppm: 177.8, 172.5, 80.3, 72.4, 69.0, 51.7, 51.3, 50.7, 50.4, 50.1, 41.6, 41.4, 41.1,
40.8, 40.1, 39.9, 38.3, 37.9, 37.0, 36.4, 33.0, 31.9, 31.1, 29.8, 29.1, 26.3, 23.9, 23.3, 23.1, 22.9, 22.6, 21.4,
17.1, 16.5

| | | | | |
|---|---|---|---|---|
| 23/123 | A | Tetrahydrofusidic acid-N-succinimide ester | N,N′-bis(4-amino- butyl)-1,3-propane- diamine | 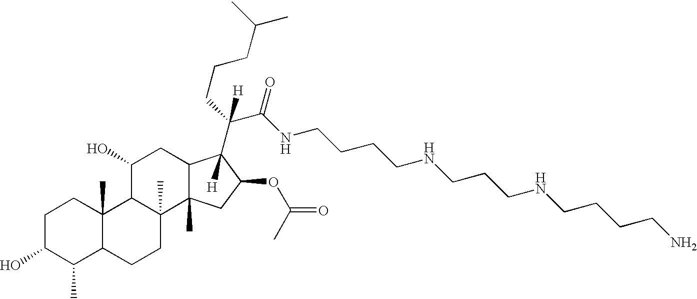 |

¹³C NMR (CD₃OD), δ/ppm: 177.5, 172.6, 80.1, 72.5, 69.0, 51.4, 51.3, 50.3, 50.1, 48.0, 41.6, 41.4, 41.1,
40.8, 40.1, 40.0, 38.3, 37.9, 37.0, 36.3, 33.0, 31.8, 31.1, 29.1, 27.8, 27.8, 26.7, 26.6, 26.3, 23.9, 23.2, 23.1,
23.0, 22.6, 21.4, 17.0, 16.5

| | | | | |
|---|---|---|---|---|
| 24/124 | A | Tetrahydrofusidic acid-N-succinimide ester | N,N′-bis(3-amino- propyl)-ethylene- diamine | 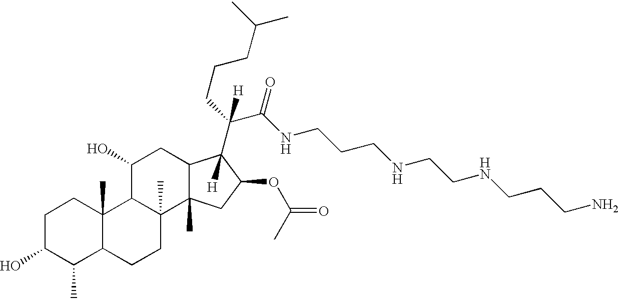 |

¹³C NMR (CD₃OD), δ/ppm: 177.5, 172.6, 80.2, 72.4, 68.9, 51.5, 51.3, 50.3, 50.1, 48.3, 41.6, 41.3, 41.1,
40.5, 40.1, 38.3, 37.9, 37.1, 36.4, 33.1, 32.4, 31.8, 31.1, 30.3, 29.2, 26.4, 23.9, 23.3, 23.2, 23.0, 22.6, 21.4,
17.1, 16.5

| | | | |
|---|---|---|---|
| 25/125 | A | Tetrahydrofusidic acid-N-succinimide ester | 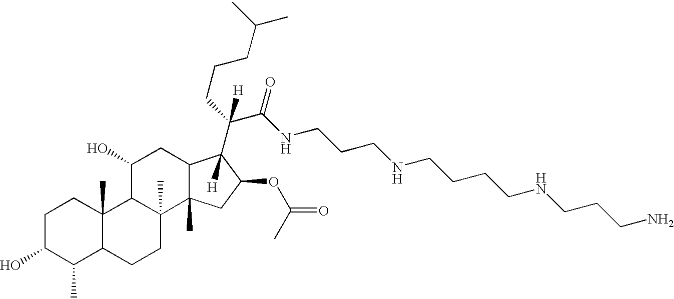 |

¹³C NMR (CD₃OD), δ/ppm: ¹³C NMR: (CD₃OD) 177.6, 172.5, 80.2, 72.4, 68.9, 51.5, 51.2, 50.3, 50.1,
48.1, 47.9, 41.5, 41.3, 41.1, 40.4, 40.1, 38.3, 38.1, 37.9, 37.0, 36.4, 33.0, 31.8, 31.3, 31.0, 29.8, 29.1, 28.0,
27.8, 26.4, 23.9, 23.3, 23.2, 23.0, 22.6, 21.4, 17.1, 16.5
MS (direct inlet) m/z = 705.59 (M + H)⁺, 604.70, 344.44, 305.42, 225.20, 203.21

| | | | | |
|---|---|---|---|---|
| 26/126 | A | Tetrahydrofusidic acid-N-succinimide ester | 6,6′-diamino-di- hexylamine | 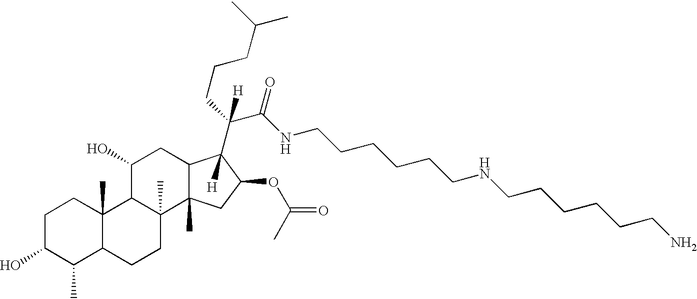 |

¹³C NMR (CD₃OD), δ/ppm: 177.4, 172.6, 80.1, 72.5, 69.0, 51.4, 51.3, 50.7, 50.3, 50.1, 42.4, 41.6, 41.4,
41.1, 40.3, 40.1, 38.3, 37.9, 37.0, 36.3, 33.3, 33.1, 31.7, 31.0, 30.3, 29.2, 28.3, 28.2, 28.1, 27.9, 26.4, 23.9,
23.3, 23.1, 23.0, 22.6, 21.4, 17.1, 16.5

| | | | | |
|---|---|---|---|---|
| 27/127 | A | Tetrahydrofusidic acid-N-succinimide ester | 8,8′-diamino-dioctyl- amine | 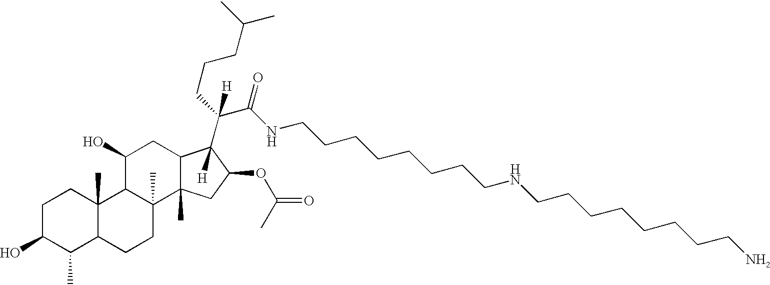 |

¹³C NMR (CD₃OD), δ/ppm: 177.3, 172.6, 80.1, 72.5, 69.0, 51.4, 51.3, 50.7, 50.3, 50.1, 42.4, 41.5, 41.4,
41.1, 40.3, 40.1, 38.3, 37.9, 37.0, 36.3, 33.3, 33.1, 31.7, 31.0, 30.6, 30.6, 30.5, 30.4, 30.3, 30.3, 29.2, 28.4,
28.2, 28.0, 26.4, 23.9, 23.3, 23.2, 23.0, 22.6, 21.5, 17.1, 16.5

| | | | | |
|---|---|---|---|---|
| 28/128 | A | Tetrahydrofusidic acid-N-succinimide ester | N,N′-bis(3-amino- propyl)-1,3-propane- diamine | 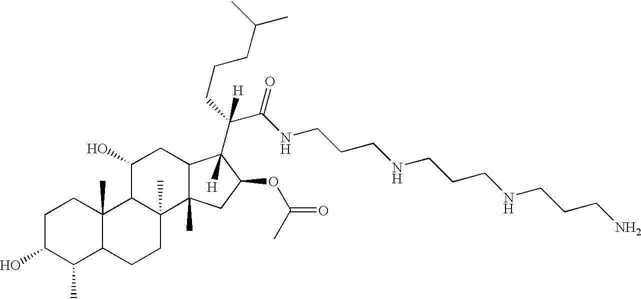 |

¹³C NMR (CD₃OD), δ/ppm: 177.8, 172.6, 80.1, 72.5, 68.9, 51.3, 50.3, 50.1, 47.7, 47.6, 41.5, 41.4, 41.1,
40.1, 39.9, 38.3, 38.0, 37.0, 36.4, 33.1, 31.8, 31.1, 29.1, 29.0, 29.0, 28.0, 26.4, 23.9, 23.3, 23.2, 23.0, 22.6,
21.4, 17.1, 16.5

| | | | | |
|---|---|---|---|---|
| 29/129 | A | Tetrahydrofusidic acid-N-succinimide ester | tetra- ethylene-pentamine | 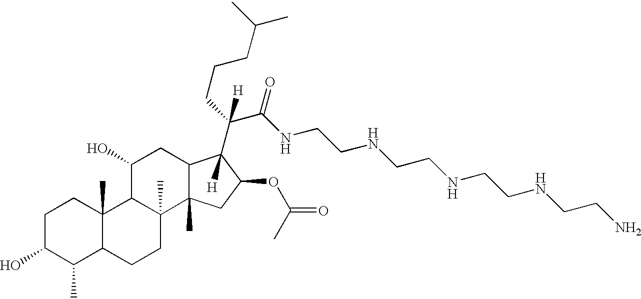 |

¹³C NMR (CD₃OD), δ/ppm: 178.2, 173.0, 80.9, 72.9, 69.3, 52.3, 51.7, 51.5, 50.8, 50.5, 48.4, 42.1, 41.8,
41.7, 41.5, 40.5, 40.5, 38.7, 38.4, 37.5, 36.9, 33.5, 32.4, 31.5, 29.5, 26.7, 24.3, 23.8, 23.6, 23.3, 23.0, 21.8,
17.6, 16.9

| | | | | |
|---|---|---|---|---|
| 30/130 | A | 11-desoxy-tetrahydro fusidic acid N-succinimide ester | 1,6-hexanediamine | 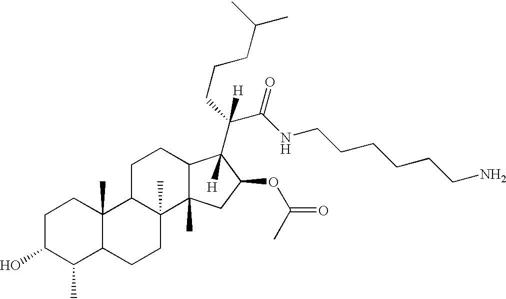 |

NMR (CD₃OD), δ/ppm: 177.3, 172.6, 80.7, 72.4, 52.1, 50.8, 50.6, 46.6, 45.9, 42.4, 41.0, 40.4, 40.3, 40.0,
38.9, 37.4, 36.4, 34.4, 33.4, 32.2, 31.1, 30.3, 30.0, 29.1, 28.1, 27.8, 26.8, 26.3, 24.5, 23.1, 22.9, 21.6, 21.3,
21.3, 20.7, 17.8, 16.5

| | | | | |
|---|---|---|---|---|
| 31/131 | A | 11-desoxy-tetrahydro fusidic acid N-succinimide ester | 3,3′-diamino-di- propylamine | 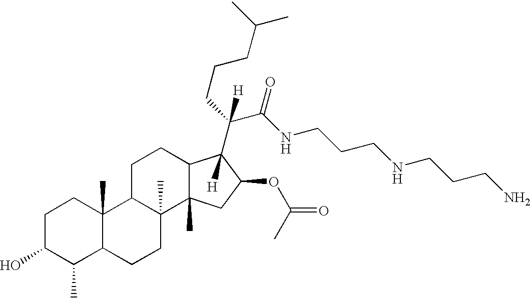 |

¹³C NMR (CD₃OD), δ/ppm: 177.5, 172.6, 80.5, 72.4, 52.0, 50.9, 50.5, 46.6, 45.9, 41.0, 40.6, 40.4, 40.0,
39.0, 38.3, 37.4, 36.3, 34.5, 33.0, 32.1, 31.1, 30.1, 30.0, 29.1, 26.8, 26.4, 24.5, 23.1, 22.9, 21.6, 21.3, 21.3,
20.7, 17.8, 16.5

| | | | | |
|---|---|---|---|---|
| 32/132 | A | 11-desoxy-tetrahydro fusidic acid N-succinimide ester | diethylenetriamine | 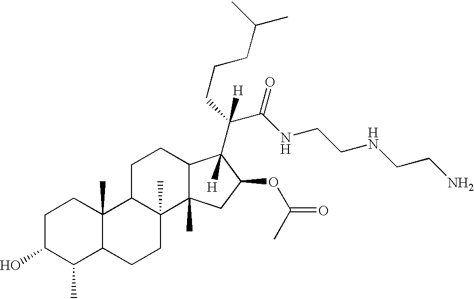 |

¹³C NMR (CD₃OD), δ/ppm: 177.6, 172.6, 80.4, 72.5, 52.0, 51.9, 50.9, 50.5, 46.5, 45.9, 41.8, 41.0, 40.4,
40.0, 39.0, 37.4, 36.4, 34.5, 32.0, 31.1, 29.9, 29.1, 26.8, 26.3, 24.4, 231, 22.9, 21.6, 2.31, 21.3, 20.7, 17.8,
16.5

| | | | | |
|---|---|---|---|---|
| 33/133 | A | 11-desoxy-tetrahydro fusidic acid N-succinimide ester | N,N′-bis(2-amino- ethyl)-1,3-propane- diamine | 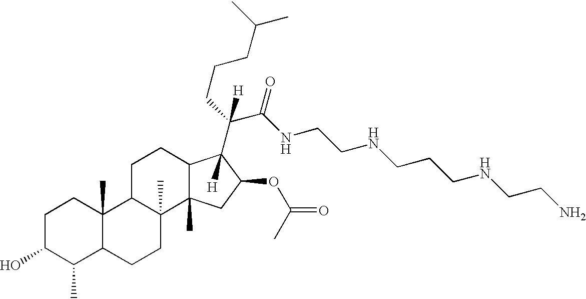 |

¹³C NMR (CD₃OD), δ/ppm: 177.7, 172.6, 80.4, 72.4, 52.4, 51.9, 50.8, 50.5, 49.8, 48.7, 48.6, 46.6, 45.9,
41.6, 41.0, 40.4, 40.0, 39.9, 39.0, 37.4, 36.4, 34.5, 32.1, 31.1, 30.5, 30.0, 29.1, 26.8, 26.3, 24.5, 23.1, 22.9,
21.6, 21.3, 21.4, 20.7, 17.8, 16.5

| | | | | |
|---|---|---|---|---|
| 34/134 | A | 11-desoxy-tetrahydro fusidic acid N-succinimide ester | tetraethylene-tetramine | 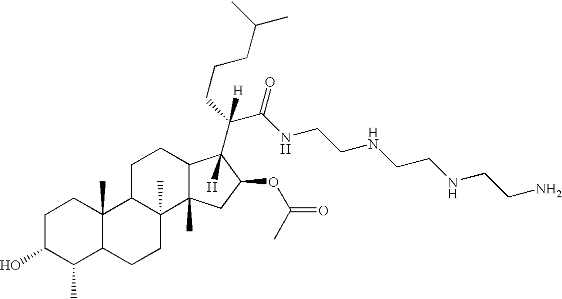 |

¹³C NMR (CD₃OD), δ/ppm: 177.6, 172.6, 80.4, 72.4, 52.4, 51.9, 50.8, 50.5, 46.5, 45.9, 41.8, 41.0, 40.4,
40.0, 38.9, 37.4, 36.3, 34.5, 32.0, 31.1, 30.0, 29.1, 26.8, 26.3, 24.4, 23.1, 22.9, 21.6, 21.3, 21.3, 20.7, 17.8,
16.5

| | | | | |
|---|---|---|---|---|
| 35/135 | A | 11-desoxy-tetrahydro fusidic acid N-succinimide ester | spermine | 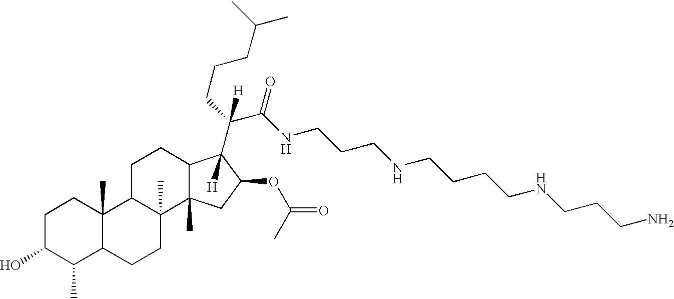 |

¹³C NMR (CD₃OD), δ/ppm: 177.5, 172.6, 80.5, 72.4, 52.0, 50.8, 50.6, 50.5, 50.5, 46.6, 45.9, 41.0, 40.6,
40.4, 40.0, 38.9, 38.3, 37.4, 36.3, 34.5, 32.7, 32.1, 31.1, 30.1, 30.0, 29.1, 28.3, 28.2, 26.8, 26.4, 24.2, 23.1,
22.9, 21.6, 21.3, 21.3, 20.7, 17.8, 16.5

| | | | | |
|---|---|---|---|---|
| 36/136 | A | 11-desoxy-tetrahydro fusidic acid N-succinimide ester | tetra- ethylene-pentamine | 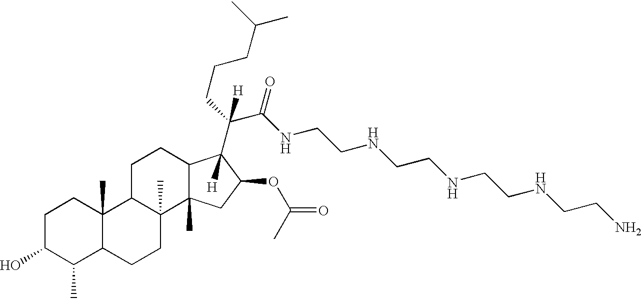 |

¹³C NMR (CD₃OD), δ/ppm: 178.1, 173.0, 80.8, 72.8, 52.3, 51.2, 50.9, 46.9, 46.3, 41.4, 40.8, 40.4, 39.4,
37.8, 36.8, 34.9, 32.5, 31.5, 30.4, 29.5, 27.2, 26.7, 24.9, 23.5, 23.3, 22.0, 21.8, 21.7, 21.1, 18.2, 16.9

| | | | | |
|---|---|---|---|---|
| 37/137 | A | 11-desoxy-tetrahydro fusidic acid N-succinimide ester | spermidine | 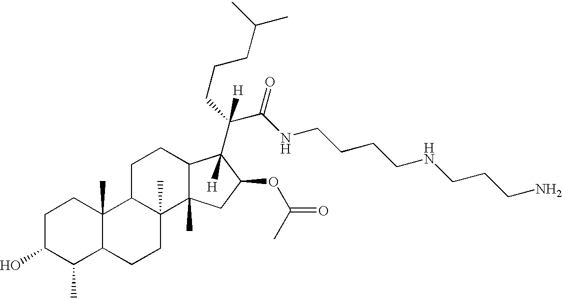 |

¹³C NMR (CD₃OD), δ/ppm: 177.5, 172.6, 80.6, 72.4, 52.0, 50.9, 50.6, 50.5, 46.6, 45.9, 42.3, 41.0, 40.4,
40.0, 39.0, 38.3, 37.4, 36.3, 34.5, 32.1, 31.3, 31.1, 30.1, 30.0, 29.1, 27.9, 26.8, 26.4, 24.5, 23.1, 22.9, 21.6,
21.3, 21.3, 20.7, 17.8, 16.5

| | | | | |
|---|---|---|---|---|
| 38/138 | A | 11-desoxy-tetrahydro fusidic acid N-succinimide ester | N,N′-bis(4-amino- butyl)-1,3-propane- diamine | 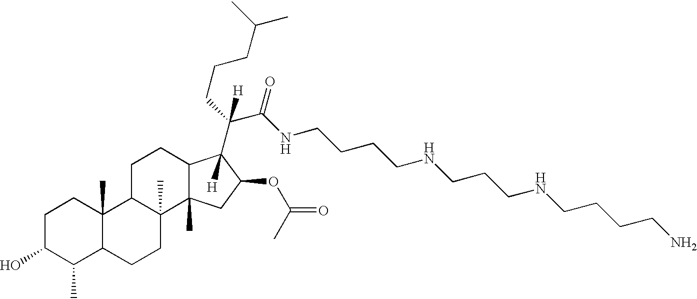 |

¹³C NMR (CD₃OD), δ/ppm: 177.1, 172.3, 80.3, 72.2, 51.7, 50.6, 50.3, 50.0, 50.0, 46.3, 45.6, 41.8, 40.8,
40.2, 39.9, 39.8, 38.7, 37.2, 36.1, 34.2, 31.9, 30.9, 30.5, 29.7, 29.6, 28.9, 27.9, 27.7, 27.5, 26.5, 26.1, 24.2,
22.9, 22.7, 21.3, 21.1, 21.1, 20.5, 17.6, 16.3

| | | | | |
|---|---|---|---|---|
| 39/139 | A | 16(17)-en-16-desacetoxy-tetrahydro fusidic acid-N-succinimide ester | spermine | 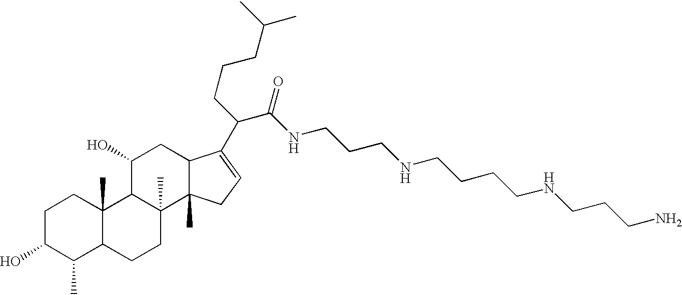 |

¹³C NMR (CD₃OD), δ/ppm: (1:1 mixture of C-20 epimers)
176.5, 176.3, 145.7, 145.6, 125.7, 125.3, 72.5, 69.3, 69.2, 55.1, 55.0, 51.9, 51.8, 50.4, 50.4, 47.8, 47.7,
44.8, 44.4, 41.4, 41.4, 40.6, 40.5, 40.0, 39.8, 39.7, 38.3, 38.0, 37.9, 37.4, 34.0, 34.0, 33.4, 32.4, 31.3,
31.2, 30.2, 30.2, 29.2, 28.1, 26.9, 26.8, 25.3, 25.3, 23.5, 23.4, 23.1, 23.0, 23.0, 22.4, 18.1, 18.0, 16.6
MS (direct inlet) m/z = 645.58 (M + H)⁺, 569.53, 321.54, 305.55, 203.21

| | | | | |
|---|---|---|---|---|
| 40/140 | A | 16-Desacetoxy tetrahydrofusidic acid-N-succinimide ester | 1,6-hexanediamine | 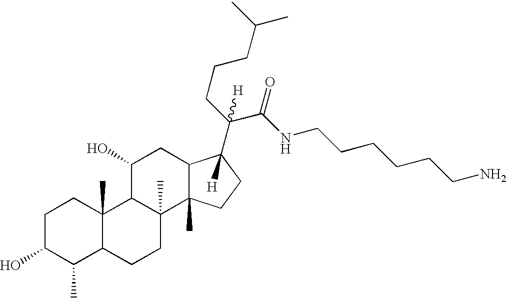 |

¹³C NMR (CD₃OD), δ/ppm: 178.0, 72.6, 69.5, 52.3, 51.8, 51.7, 43.9, 42.0, 41.7, 40.2, 40.1, 38.2, 38.0,
37.5, 37.1, 33.2, 33.0, 32.1, 31.4, 31.1, 30.6, 29.2, 27.8, 27.4, 26.9, 26.6, 23.8, 23.3, 23.1, 23.0, 22.7, 16.6,
16.4

| | | | | |
|---|---|---|---|---|
| 41/141 | A | 16-Desacetoxy tetrahydrofusidic acid-N-succinimide ester | 1,6-hexanediamine | 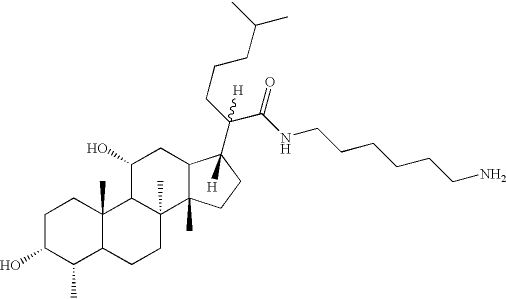 |

¹³C NMR (CD₃OD), δ/ppm: 178.5, 72.5, 69.4, 53.9, 51.8, 51.7, 43.8, 42.4, 42.2, 41.5, 40.2, 40.1, 38.3,
37.9, 37.1, 36.7, 33.1, 33.0, 32.5, 31.6, 31.1, 31.1, 30.3, 29.2, 28.5, 28.0, 27.6, 26.5, 23.9, 23.2, 23.2, 22.9,
22.7, 16.6, 16.4
Comment: C-20 epimer of Compound 137.

| | | | | |
|---|---|---|---|---|
| 42/142 | A | 16-Desacetoxy tetrahydrofusidic acid-N-succinimide ester | diethylenetriamine | 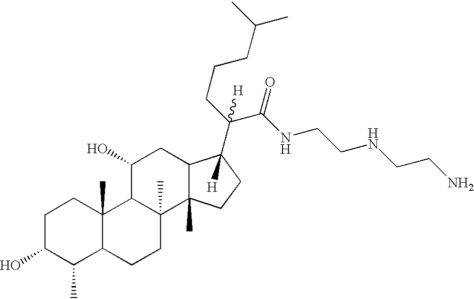 |

¹³C NMR (CD₃OD), δ/ppm: 178.9, 72.5, 69.3, 54.5, 51.9, 51.7, 43.7, 42.6, 41.6, 41.5, 40.1, 38.2, 38.0,
37.1, 36.9, 33.1, 32.9, 31.7, 31.2, 31.1, 29.1, 28.9, 26.5, 23.8, 23.3, 23.2, 22.9, 22.6, 16.6, 16.5

| | | | | |
|---|---|---|---|---|
| 43/143 | A | 16-Desacetoxy tetrahydrofusidic acid-N-succinimide ester | 3,3′-diamino-dipropyl- amine | 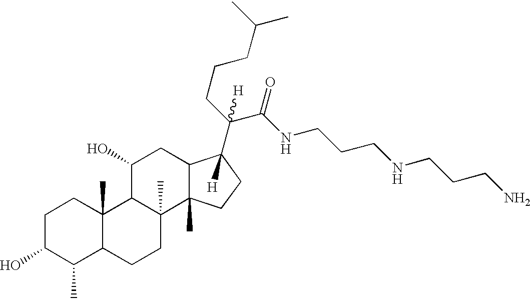 |

¹³C NMR (CD₃OD), δ/ppm: 178.7, 72.5, 69.3, 54.1, 51.8, 51.7, 48.1, 43.8, 42.4, 41.5, 40.6, 40.2, 38.3,
38.2, 38.0, 37.1, 36.8, 33.1, 32.7, 32.6, 31.7, 31.2, 31.1, 30.1, 29.2, 28.6, 26.6, 23.8, 23.2, 22.9, 22.7, 16.6,
16.4

| | | | | |
|---|---|---|---|---|
| 44/144 | A | 16-Desacetoxy tetrahydrofusidic acid-N-succinimide ester | spermidine | 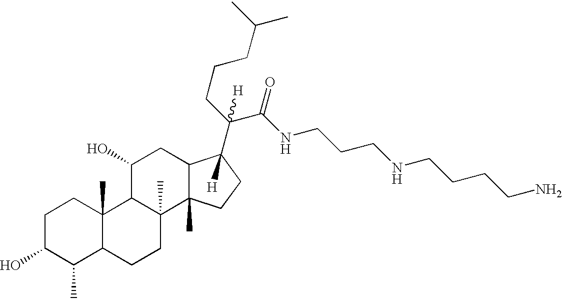 |

¹³C NMR (CD₃OD), δ/ppm: 178.7, 72.5, 69.3, 54.0, 51.8, 51.7, 50.3, 47.9, 43.8, 42.4, 42.0, 41.5, 40.2,
38.3, 38.1, 38.0, 37.1, 36.8, 33.1, 32.5, 31.6, 31.2, 31.1, 30.5, 30.0, 29.2, 28.6, 27.8, 26.6, 23.8, 23.2, 22.9,
22.7, 16.6, 16.4

| | | | | |
|---|---|---|---|---|
| 45/145 | A | 16-Desacetoxy tetrahydrofusidic acid-N-succinimide ester | N,N′-bis(3-amino- propyl)-1,3-propane- diamine | 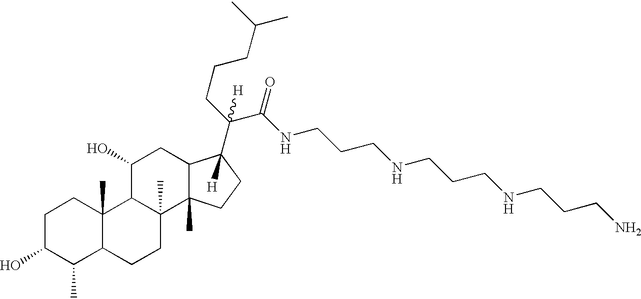 |

¹³C NMR (CD₃OD), δ/ppm: 178.7, 72.5, 69.3, 54.0, 51.8, 51.7, 48.1, 43.9, 42.4, 41.5, 40.5, 40.1, 38.3,
38.2, 38.0, 37.1, 36.8, 33.1, 32.5, 31.8, 31.7, 31.2, 31.1, 30.1, 29.7, 29.2, 28.6, 26.6, 23.8, 23.2, 22.9, 22.7,
16.6, 16.4

| | | | | |
|---|---|---|---|---|
| 46/146 | A | 16-Desacetoxy tetrahydrofusidic acid-N-succinimide ester | spermine | 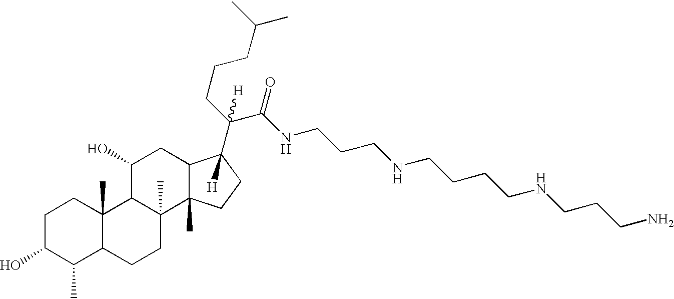 |

¹³C NMR (CD₃OD), δ/ppm: 178.9, 72.5, 69.3, 53.9, 51.8, 51.7, 50.1, 48.0, 47.7, 43.9, 42.4, 41.5, 40.3,
40.2, 38.3, 38.0, 37.1, 36.8, 33.1, 32.5, 31.7, 31.2, 31.1, 30.4, 29.6, 29.2, 28.6, 27.6, 26.6, 23.8, 23.2, 23.0,
22.7, 16.6, 16.4

| | | | | |
|---|---|---|---|---|
| 47/147 | A | 16-Desacetoxy tetrahydrofusidic acid-N-succinimide ester | tetra- ethylene-pentamine | 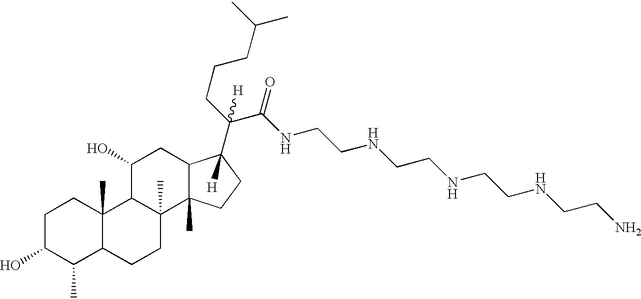 |

¹³C NMR (CD₃OD), δ/ppm: 178.9, 72.5, 69.3, 54.4, 51.8, 51.1, 43.6, 42.6, 41.5, 41.3, 40.1, 38.2, 38.0,
37.1, 33.1, 32.9, 31.7, 31.2, 31.1, 29.1, 28.9, 26.5, 23.8, 23.4, 23.2, 22.9, 22.6, 16.6, 16.5

| | | | | |
|---|---|---|---|---|
| 48/148 | A | 16-Desacetoxy tetrahydrofusidic acid-N-succinimide ester | tetra- ethylene-pentamine | 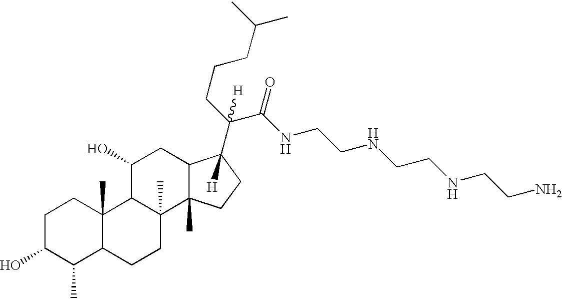 |

¹³C NMR (CD₃OD), δ/ppm: 178.9, 72.5, 69.3, 54.5, 51.9, 51.7, 51.3, 43.6, 42.6, 41.6, 41.4, 40.1, 38.3,
38.0, 37.1, 36.9, 33.0, 32.9, 31.7, 31.2, 31.1, 29.1, 28.9, 26.5, 23.9, 23.3, 23.2, 22.9, 22.7, 16.6, 16.4

| | | | |
|---|---|---|---|
| 49/149 | A | N,N′-bis(2-amino- ethyl)-1,3-propane- diamine | 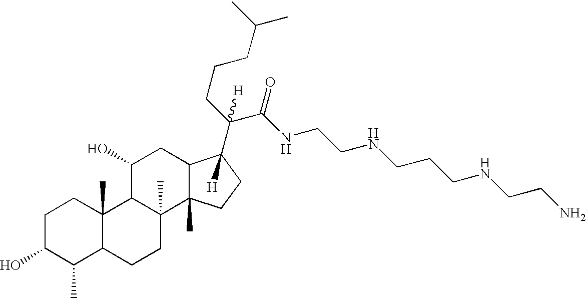 |

¹³NMR (CD₃OD), δ/ppm: 178.9, 72.5, 69.3, 54.4, 52.1, 51.8, 51.7, 48.7, 48.6, 43.7, 42.6, 41.6, 41.4,
40.1, 40.0, 38.3, 38.0, 37.1, 36.9, 33.1, 32.8, 31.7, 31.2, 31.1, 30.2, 29.1, 28.8, 26.5, 23.9, 23.3, 23.2, 22.9,
22.7, 16.6, 16.4

| | | | | |
|---|---|---|---|---|
| 50/150 | A | 11-desoxy-16-desacet- oxy-17S,20,24,25-tetra- hydrofusidic acid N-succinimide ester | 1,6-hexanediamine | 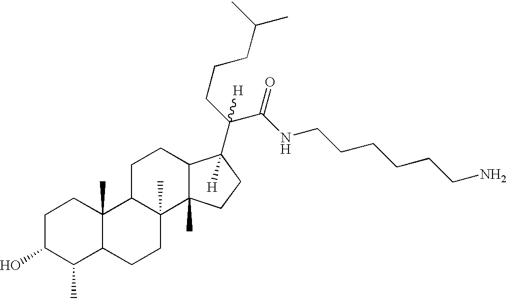 |

¹³C NMR (CD₃OD), δ/ppm: 178.4, 72.5, 54.7, 52.0, 47.5, 46.4, 44.5, 42.4, 40.6, 40.2, 40.1, 39.0, 37.5,
36.4, 34.6, 33.4, 33.0, 31.7, 31.1, 30.3, 30.0, 29.2, 29.0, 28.1, 27.8, 27.3, 26.5, 24.7, 23.2, 22.9, 21.5, 21.4,
2.7, 17.2, 16.6

| | | | | |
|---|---|---|---|---|
| 51/151 | A | 11-desoxy-16-desace- toxy-17S,20,24,25-tetra- hydrofusidic acid N-succinimide ester | 3,3′-diamino-di- propylamine | 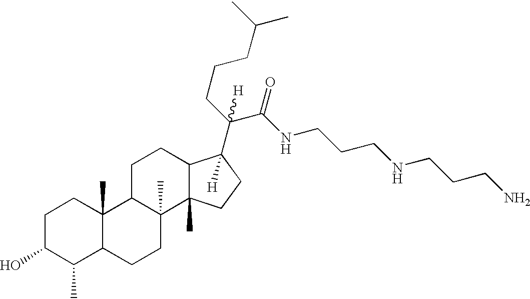 |

¹³C NMR (CD₃OD), δ/ppm: 178.6, 72.5, 54.5, 52.0, 47.5, 46.4, 44.5, 40.7 40.1, 39.0, 38.2, 37.5, 36.3,
34.6, 33.0, 32.8, 31.7, 31.2, 30.1, 30.0, 29.1, 28.8, 27.3, 26.6, 24.7, 23.2, 22.9, 21.5, 21.5, 20.7, 17.2, 16.6

| | | | | |
|---|---|---|---|---|
| 52/152 | A | 11-desoxy-16-desace- toxy-17S,20,24,25-tetra- hydrofusidic acid N-succinimide ester | N,N′-bis(2-amino- ethyl)-1,3-propane- diamine | 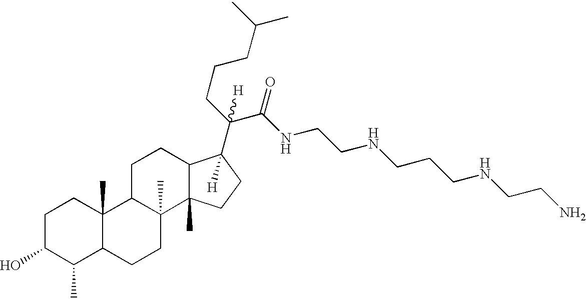 |

¹³C NMR (CD₃OD), δ/ppm: 178.8, 72.5, 54.6, 52.3, 52.0, 47.5, 46.4, 44.5, 41.6, 40.7, 40.1, 39.8, 39.0,
37.5, 36.4, 34.6, 32.9, 31.7, 31.2, 30.5, 30.0, 29.1, 28.8, 27.3, 26.5, 24.7, 23.2, 22.9, 21.5, 21.4, 20.7, 17.2,
16.6

| | | | | |
|---|---|---|---|---|
| 53/153 | A | 11-desoxy-16-desacet- oxy-17S,20,24,25-tetra- hydrofusidic acid N-succinimide ester | N,N′-bis(3-amino- propyl)-ethylene- diamine | 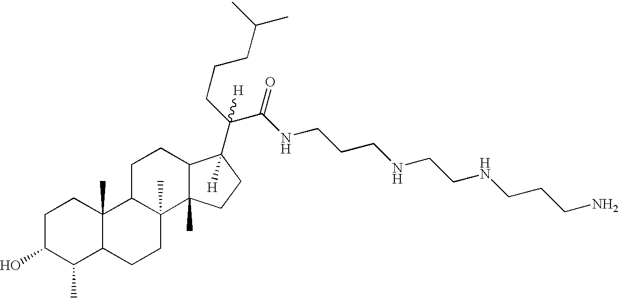 |

¹³C NMR (CD₃OD), δ/ppm: 178.6, 72.5, 54.5, 52.0, 49.7, 48.3, 47.5, 46.4, 44.5, 40.7, 40.6, 40.1, 39.0,
38.2, 37.5, 36.4, 34.6, 32.8, 31.7, 31.2, 30.3, 30.0, 29.1, 28.8, 27.3, 26.6, 24.7, 23.2, 22.9, 21.5, 21.4, 20.7
17.2, 16.6

| | | | | |
|---|---|---|---|---|
| 54/154 | A | 11-desoxy-16-desacet- oxy-17S,20,24,25-tetra- hydrofusidic acid N-succinimide ester | spermidine | 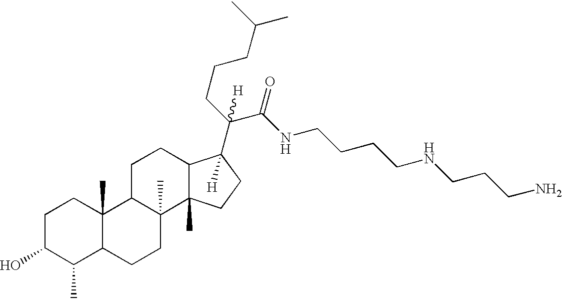 |

¹³C NMR (CD₃OD), δ/ppm: 178.7, 72.5, 54.5, 52.0, 50.5, 48.1, 47.5, 46.4, 44.5, 42.1, 40.6, 40.1, 39.0,
38.2, 37.5, 36.3, 34.6, 32.8, 31.7, 31.2, 30.8, 30.0, 29.2, 28.8, 27.9, 27.3, 26.6, 24.7, 23.2, 22.9, 21.5, 21.4,
20.7, 17.2, 16.6

| | | | | |
|---|---|---|---|---|
| 55/155 | A | 11-desoxy-16-desace- toxy-17S,20,24,25-tetra- hydrofusidic acid N-succinimide ester | N,N′-bis(3-amino- propyl)-1,3-propane- diamine | 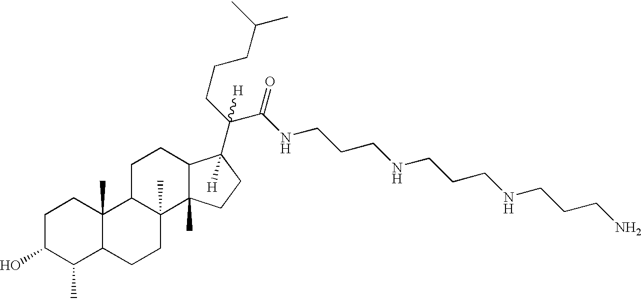 |

¹³C NMR (CD₃OD), δ/ppm: 178.6, 72.5, 54.5, 52.0, 48.9, 48.3, 47.4, 46.3, 44.5, 40.6, 40.5, 40.1, 39.0,
38.2, 37.5, 36.3, 34.6, 32.8, 32.4, 31.7, 31.2, 30.1, 30.0, 29.9, 29.1, 28.8, 27.3, 26.5, 24.7, 23.2, 22.9, 21.5,
21.4, 20.7, 17.2, 16.6

| | | | | |
|---|---|---|---|---|
| 56/156 | A | 11-desoxy-16-desace- toxy-17S,20,24,25-tetra- hydrofusidic acid N-succinimide ester | spermine | 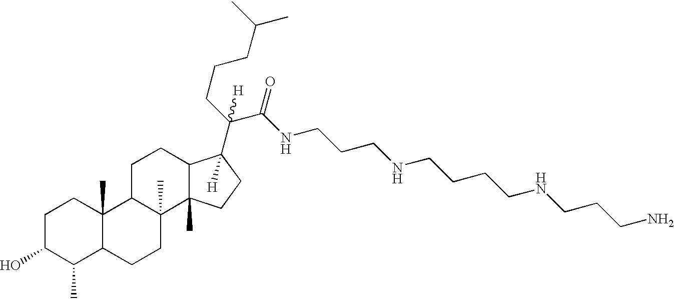 |

¹³C NMR (CD₃OD), δ/ppm: 178.7, 72.5, 54.5, 52.0, 50.6, 50.4, 48.1, 47.4, 46.4, 44.5, 40.6, 40.6, 40.1,
39.0, 38.2, 37.5, 36.3, 34.6, 32.8, 32.4, 31.7, 31.2, 30.0, 29.1, 28.8, 28.3, 28.1, 27.3, 26.6, 24.7, 23.2, 22.9,
21.5, 21.4, 20.7, 17.2, 16.6

| | | | | |
|---|---|---|---|---|
| 57/157 | A | 11-desoxy-16-desacet- oxy-17S,20,24,25-tetra- hydrofusidic acid N-succinimide ester | N,N′-bis(4-amino- butyl)-1,3-propane- diamine | 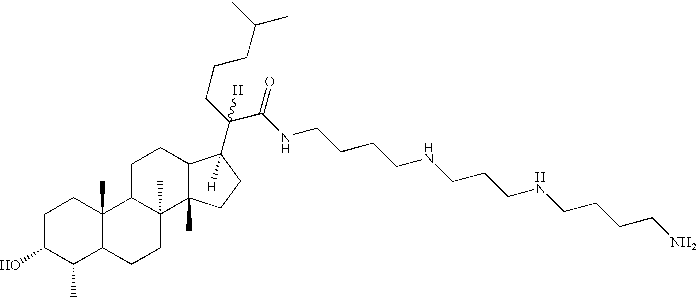 |

¹³C NMR (CD₃OD), δ/ppm: 178.5, 72.5, 54.6, 52.0, 50.4, 50.3, 47.5, 46.4, 44.5, 42.2, 40.6, 40.1, 39.0,
37.5, 36.3, 34.6, 32.9, 31.7, 31.2, 31.0, 30.0, 30.0, 29.1, 28.9, 28.2, 28.0, 27.8, 27.3, 26.5, 24.7, 23.2, 22.9,
21.5, 21.4, 20.7, 17.2, 16.6

| | | | | |
|---|---|---|---|---|
| 58/158 | A | 3β-OH-tetrahydrofusidic acid-N-succinimide ester | 1,6-hexanediamine | 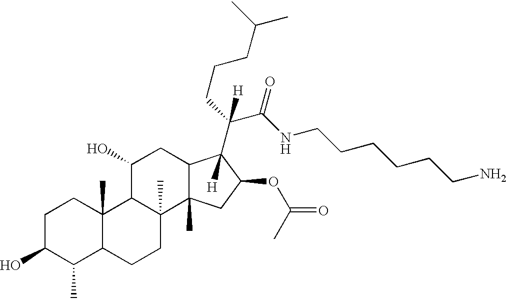 |

¹³C NMR (CD₃OD), δ/ppm: 177.3, 172.6, 80.0, 77.3, 68.8, 51.3, 51.0, 50.3, 50.1, 44.2, 42.3, 41.5, 41.3,
41.2, 41.1, 40.3, 40.1, 37.9, 37.7, 36.5, 35.2, 33.7, 33.3, 32.6, 31.7, 30.3, 29.2, 28.0, 27.7, 26.4, 24.5, 23.6,
23.1, 23.0, 22.7, 21.5, 17.1, 15.9

| | | | | |
|---|---|---|---|---|
| 59/159 | A | 3β-OH-tetrahydrofusidic acid-N-succinimide ester | diethylenetriamine | 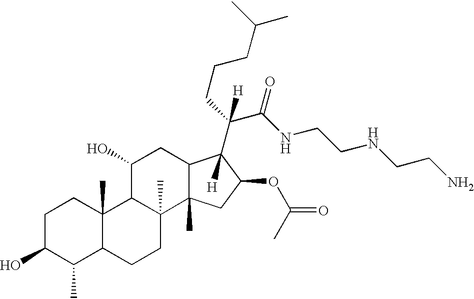 |

¹³C NMR (CD₃OD), δ/ppm: 178.1, 173.0, 80.8, 77.8, 69.1, 52.2, 51.4, 50.8, 50.6, 44.7, 42.1, 42.0, 41.7,
41.6, 41.5, 40.6, 40.5, 38.2, 37.1, 35.7, 34.1, 33.1, 32.3, 29.5, 26.7, 24.9, 24.1, 23.5, 23.2, 23.1, 21.8, 17.6,
16.4

| | | | | |
|---|---|---|---|---|
| 60/160 | A | 3β-OH-tetrahydrofusidic acid-N-succinimide ester | 3,3′-diamino-dipropyl- amino | 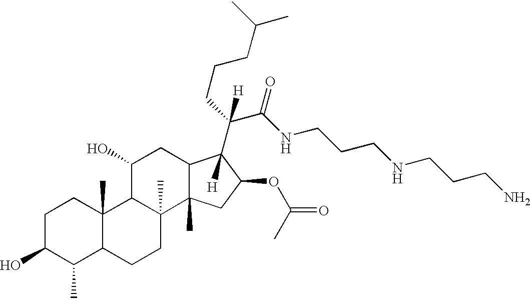 |

¹³C NMR (CD₃OD), δ/ppm: 177.5, 172.6, 80.2, 77.3, 68.7, 51.6, 50.9, 50.3, 50.2, 48.4, 44.3, 41.6, 41.3,
41.2, 41.1, 40.6, 40.1, 38.3, 37.7, 36.6, 35.3, 33.7, 33.2, 32.6, 31.8, 30.2, 29.1, 26.4, 24.5, 23.6, 23.2, 23.0,
22.7, 21.4, 17.1, 15.9

| | | | | |
|---|---|---|---|---|
| 61/161 | B | 3β-OH-tetrahydrofusidic acid-N-succinimide ester | spermidine | 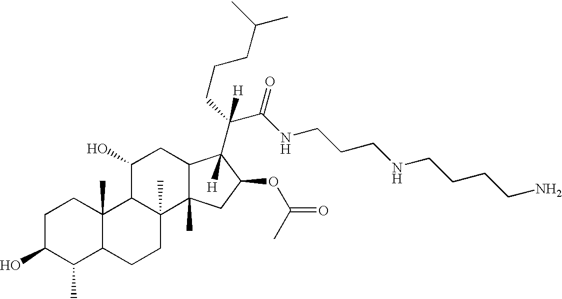 |

¹³C NMR (CD₃OD), δ/ppm:

| | | | | |
|---|---|---|---|---|
| 62/162 | B | 3β-OH-tetrahydrofusidic acid-N-succinimide ester | N,N′-bis(2-amino- ethyl)-1,3-propane- diamine | 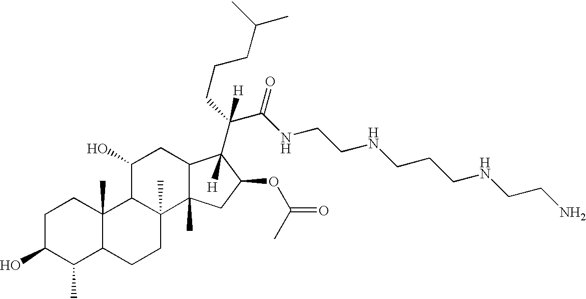 |

¹³C NMR (CD₃OD), δ/ppm: 178.1, 173.0, 80.8, 77.7, 69.2, 52.4, 52.2,
51.4, 50.8, 50.6, 49.0, 44.7, 42.0, 41.8, 41.7, 41.6, 41.5, 40.5, 40.4, 38.2, 37.0, 35.7, 34.1, 33.1, 32.3, 30.7,
29.5, 26.7, 24.9, 24.1, 23.5, 23.3, 23.1, 21.8, 17.6, 16.3

| | | | | |
|---|---|---|---|---|
| 63/163 | A | 3β-OH-tetrahydrofusidic acid-N-succinimide ester | N,N′-bis(3-amino- propyl)-1,3-propane- diamine | 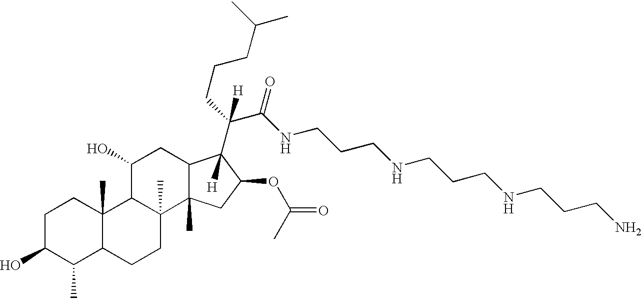 |

¹³C NMR (CD₃OD), δ/ppm: 177.5, 172.6, 80.2, 77.3, 68.7, 51.5, 50.9, 50.3, 50.2, 44.3, 41.5, 41.3, 41.2,
41.1, 40.5, 40.1, 38.2, 37.7, 36.6, 35.3, 33.7, 32.6, 32.0, 31.8, 30.1, 29.8, 29.1, 26.4, 24.5, 23.6, 23.2, 23.0,
22.7, 21.4, 17.1, 16.0

| | | | | |
|---|---|---|---|---|
| 64/164 | A | 3β-OH-tetrahydrofusidic acid-N-succinimide ester | tetra- ethylene-pentamine | 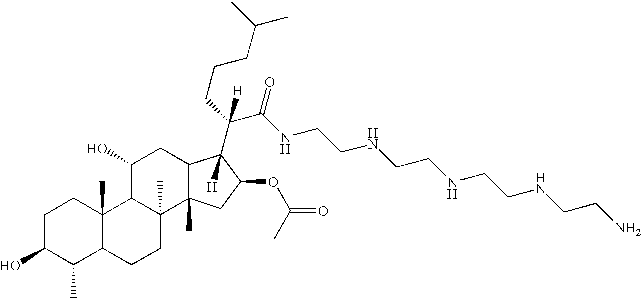 |

¹³C NMR (CD₃OD), δ/ppm: 177.7, 172.5, 80.4, 77.3, 68.7, 51.8, 51.0, 50.4, 50.1, 44.3, 41.7, 41.3, 41.2,
40.1, 37.8, 36.7, 35.3, 33.7, 32.7, 31.9, 29.1, 26.3, 24.5, 23.7, 23.1, 22.9, 22.7, 21.4, 17.2, 16.0

| | | | | |
|---|---|---|---|---|
| 65/165 | A | 3β-OH-tetrahydrofusidic acid-N-succinimide ester | N,N′-bis(4-amino- butyl)-1,3-propane- diamine | 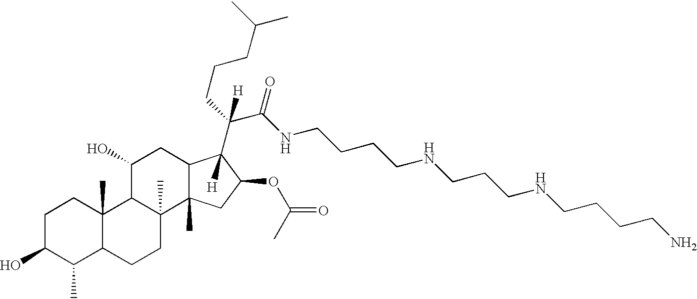 |

¹³C NMR (CD₃OD), δ/ppm:

| | | | | |
|---|---|---|---|---|
| 66/166 | A | 3β-OH-tetrahydrofusidic acid-N-succinimide ester | spermine | 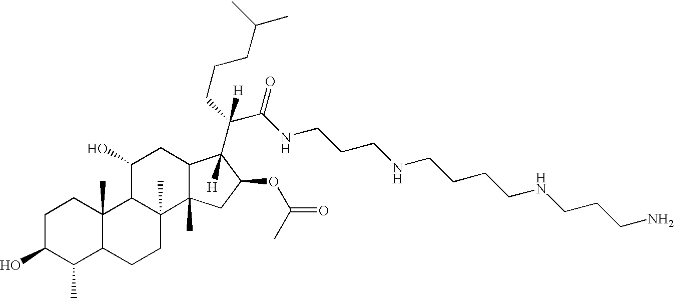 |

¹³C NMR (CD₃OD), δ/ppm: 177.5, 172.5, 80.2, 77.3, 68.7, 51.5, 50.9, 50.4, 50.3, 50.2, 47.9, 44.2, 41.5,
41.3, 41.2, 41.1, 40.4, 40.1, 38.2, 37.7, 36.6, 35.2, 33.6, 32.6, 31.7, 30.0, 29.1, 28.1, 27.9, 26.4, 24.5, 23.6,
23.2, 23.0, 22.7, 21.4, 17.1, 16.0

| | | | | |
|---|---|---|---|---|
| 67/167 | A | 3-acetoxy-tetrahydrofusidic acid | 1,6-hexanediamine | 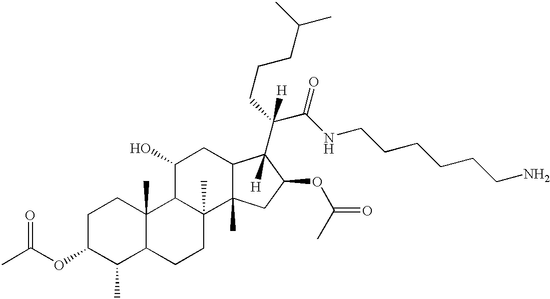 |

¹³C NMR (CD₃OD), δ/ppm: 177.3, 172.9, 172.6, 80.1, 76.2, 68.6, 51.4, 51.1, 50.3, 50.2, 42.3, 41.5, 41.3,
41.1, 40.3, 40.1, 38.9, 38.0, 36.6, 36.6, 33.5, 33.2, 31.7, 30.3, 29.2, 28.3, 28.0, 27.7, 26.4, 23.7, 23.4, 23.1,
23.0, 22.3, 21.4, 21.2, 17.2, 16.1

| | | | | |
|---|---|---|---|---|
| 68/168 | A | 3-acetoxy-tetrahydrofusidic acid | 3,3′-diamino-dipropyl- amine | 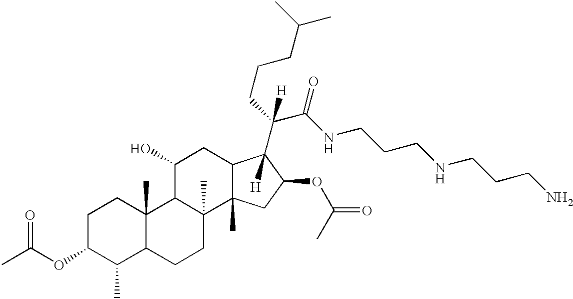 |

¹³C NMR (CD₃OD), δ/ppm: 177.5, 172.9, 172.6, 80.3, 76.2, 68.6, 51.6, 51.0, 50.4, 50.2, 48.4, 41.5, 41.3,
41.1, 40.6, 40.1, 38.9, 38.3, 38.0, 36.7, 36.6, 33.5, 33.1, 31.8, 31.7, 30.2, 29.2, 28.3, 26.4, 23.7, 23.4, 23.2,
23.0, 22.3, 21.4, 21.2, 17.2, 16.1

| | | | | |
|---|---|---|---|---|
| 69/169 | A | 3-acetoxy-tetrahydrofusidic acid | spermidine | 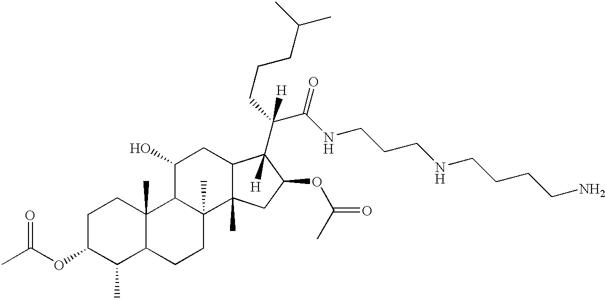 |

¹³C NMR (CD₃OD), δ/ppm: 177.5, 172.9, 172.6, 80.3, 76.2, 68.6, 51.6, 51.0, 50.5, 50.4, 50.2, 48.1, 42.3,
41.5, 41.3, 41.1, 40.1, 38.9, 38.3, 38.0, 36.7, 36.6, 33.5, 31.8, 31.7, 31.2, 30.1, 29.2, 28.3, 27.9, 26.4, 23.7,
23.4, 23.2, 23.0, 22.3, 21.4, 21.2, 17.2, 16.1

| | | | | |
|---|---|---|---|---|
| 70/170 | A | 3-acetoxy-tetrahydrofusidic acid | N,N′-bis(2-amino- ethyl)-1,3-propane- diamine | 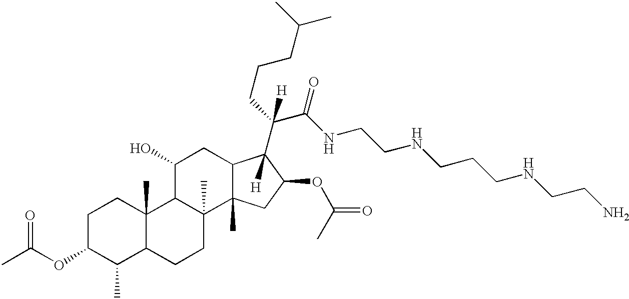 |

¹³C NMR (CD₃OD), δ/ppm: 177.7, 172.9, 172.6, 80.4, 76.2, 68.6, 52.3, 51.8, 51.1, 50.4, 50.2, 48.6, 41.6,
41.6, 41.3, 41.1, 40.1, 38.9, 38.0, 36.7, 36.6, 33.6, 31.9, 31.8, 30.4, 29.1, 28.3, 26.3, 23.8, 23.4, 23.1, 22.9,
22.2, 21.4, 21.2, 17.3, 16.1

| | | | | |
|---|---|---|---|---|
| 71/171 | A | 3-acetoxy-tetrahydrofusidic acid | N,N′-bis(3-amino- propyl)-ethylene- diamine | 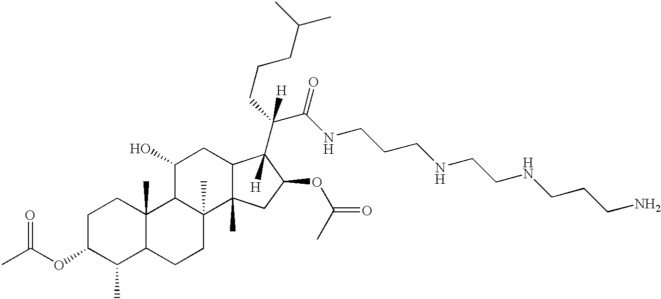 |

¹³C NMR (CD₃OD), δ/ppm: 177.5, 172.9, 172.6, 80.3, 76.2, 68.6, 51.6, 51.1, 50.4, 50.2, 49.9, 49.8, 48.4,
41.6, 41.3, 41.1, 40.6, 40.1, 38.9, 38.3, 38.0, 36.7, 36.6, 33.5, 33.0, 31.8, 31.8, 30.3, 29.2, 28.3, 26.4, 23.7,
23.4, 23.2, 23.0, 22.3, 21.4, 21.2, 17.3, 16.1

| | | | | |
|---|---|---|---|---|
| 72/172 | A | 3-acetoxy-tetrahydrofusidic acid | N,N′-bis(3-amino- propyl)-1,3-propane- diamine | 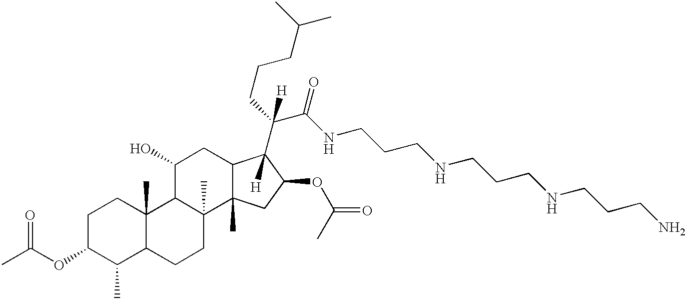 |

¹³C NMR (CD₃OD), δ/ppm: 177.5, 172.9, 172.6, 80.3, 76.2, 68.6, 51.6, 51.0, 50.4, 50.2, 48.9, 48.3, 41.6,
41.3, 41.1, 40.6, 40.1, 38.9, 38.3, 38.0, 36.7, 36.6, 33.5, 32.9, 31.9, 31.7, 30.2, 29.2, 28.3, 26.4, 23.7, 23.4,
23.2, 23.0, 22.3, 21.4, 21.2, 17.3, 16.1

| | | | | |
|---|---|---|---|---|
| 73/173 | A | 3-acetoxy-tetrahydrofusidic acid | spermine | 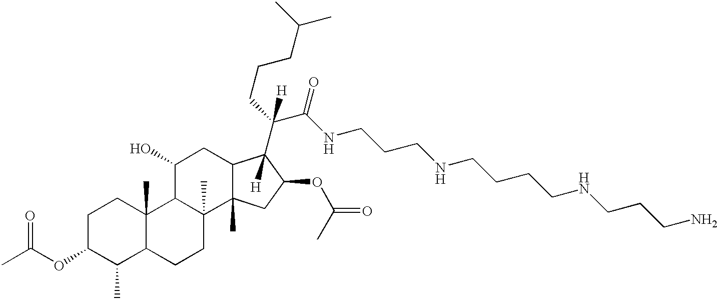 |

¹³C NMR (CD₃OD), δ/ppm: 177.5, 172.9, 172.6, 80.3, 76.2, 68.6, 51.6, 51.0, 50.6, 50.5, 50.4, 50.2, 48.1,
41.5, 41.3, 41.1, 40.6, 40.1, 38.9, 38.3, 38.0, 36.7, 36.6, 33.5, 32.7, 31.8, 31.7, 30.1, 29.2, 28.3, 28.2, 26.4,
23.7, 23.4, 23.2, 23.0, 22.3, 21.4, 21.2, 17.2, 16.1

| | | | | |
|---|---|---|---|---|
| 74/174 | A | 3-OSO₃H-11-desoxy-tetrahydrofusidic acid N-succinimide ester | 3,3′-diamino-dipropyl- amine | 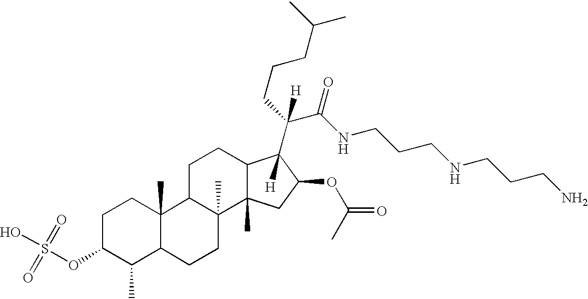 |

¹³C NMR (CD₃OD), δ/ppm: 177.5, 172.6, 81.7, 81.2, 52.3, 51.0, 50.7, 46.6, 46.1, 41.0, 40.5, 40.0, 39.9,
39.5, 38.1, 37.0, 36.2, 33.8, 32.6, 30.5, 29.5, 29.1, 28.4, 28.0, 26.9, 26.4, 24.7, 23.1, 22.9, 21.6, 21.4, 21.2,
17.8, 16.4

| | | | | |
|---|---|---|---|---|
| 75/175 | A | 3-OSO₃H-11-desoxy-tetrahydrofusidic acid N-succinimide ester | N,N′-bis(2-amino- ethyl)-1,3-propane- diamine | 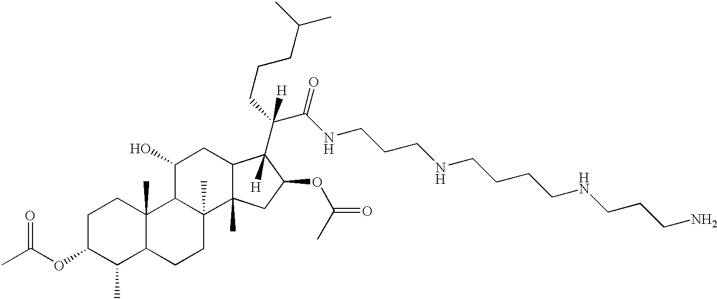 |

¹³C NMR (CD₃OD), δ/ppm: 177.7, 172.6, 81.7, 81.2, 52.5, 51.0, 50.7, 46.8, 46.1, 41.0, 40.5, 40.0, 39.7,
39.6, 37.1, 36.1, 33.9, 32.6, 30.5, 29.7, 29.1, 28.4, 27.0, 26.3, 24.7, 23.1, 22.9, 21.7, 21.4, 21.2, 21.1, 17.8,
16.4

| | | | | |
|---|---|---|---|---|
| 176/176 | A | 3-OSO₃H-11-desoxy-tetrahydrofusidic acid N-succinimide ester | spermidine | 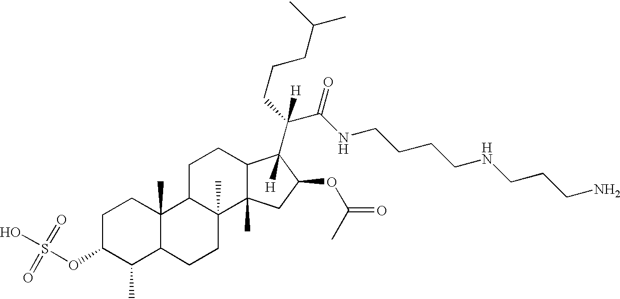 |

¹³C NMR (CD₃OD), δ/ppm: 177.5, 172.6, 81.7, 81.5, 52.6, 51.0, 50.8, 50.4, 46.7, 46.1, 40.9, 40.8, 40.5,
40.0, 39.5, 38.2, 37.1, 36.1, 33.8, 32.7, 30.5, 29.3, 29.1, 28.4, 27.3, 27.2, 27.0, 26.4, 24.7, 23.1, 22.9, 21.6,
21.4, 21.2, 21.1, 17.8, 16.4

| | | | | |
|---|---|---|---|---|
| 77/177 | A | 3-OSO₃H-11-desoxy-tetrahydrofusidic acid N-succinimide ester | spermine | 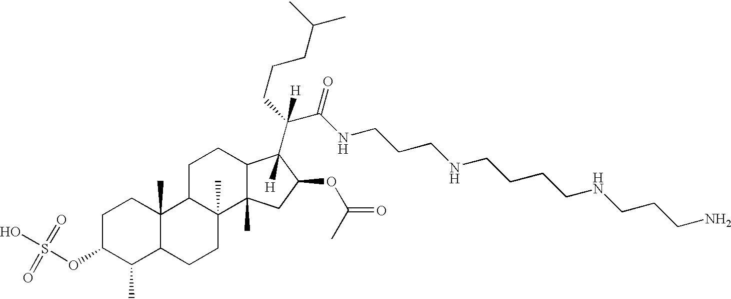 |

¹³C NMR (CD₃OD), δ/ppm: 177.5, 172.6, 81.7, 81.5, 52.6, 51.0, 50.8, 50.6, 47.8, 46.7, 46.0, 40.9, 40.5,
40.0, 40.0, 39.6, 38.2, 37.1, 36.1, 33.9, 32.8, 30.5, 29.1, 28.7, 28.5, 27.6, 27.1, 27.0, 26.4, 24.7, 23.1, 22.9,
21.6, 21.3, 21.2, 21.1, 17.8, 16.4

| | | | | |
|---|---|---|---|---|
| 78/178 | A | 3-OSO₃H-11-desoxy-tetrahydrofusidic acid N-succinimide ester | tetra- ethylene-pentamine | 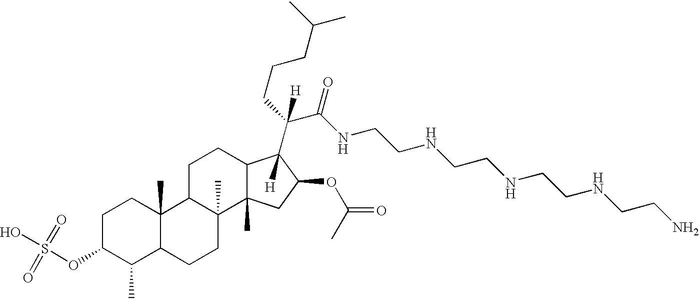 |

NMR (CD₃OD), δ/ppm: 177.6, 172.6, 81.7, 81.1, 53.6, 52.3, 50.9, 50.7, 46.6, 46.1, 40.9, 40.5, 40.0, 39.6,
37.1, 36.1, 33.9, 32.6, 30.4, 29.1, 28.4, 27.0, 26.3, 24.7, 23.1, 23.0, 22.9, 21.7, 21.4, 21.1, 21.1, 17.8, 16.4

| | | | | |
|---|---|---|---|---|
| 79/179 | A | 3-OSO₃H-11-desoxy-tetrahydrofusidic acid N-succinimide ester | N,N′-bis(3-amino- propyl)-ethylene- diamine | 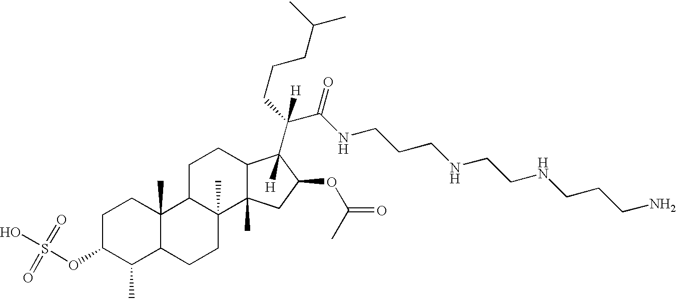 |

¹³C NMR (CD₃OD), δ/ppm: 177.5, 172.6, 81.7, 81.1, 52.3, 50.9, 50.7, 48.0, 46.6, 46.0, 40.9, 40.5, 40.0,
39.6, 38.1, 37.1, 36.1, 33.9, 32.5, 30.4, 29.6, 29.1, 28.4, 28.3, 26.9, 26.4, 24.7, 23.1, 22.9, 21.6, 21.3, 21.2,
21.1, 17.8, 16.4

| | | | | |
|---|---|---|---|---|
| 80/180 | A | 3-OSO₃H-11-desoxy-tetrahydrofusidic acid N-succinimide ester | N,N′-bis(3-amino- propyl)-1,3-propane- diamine | 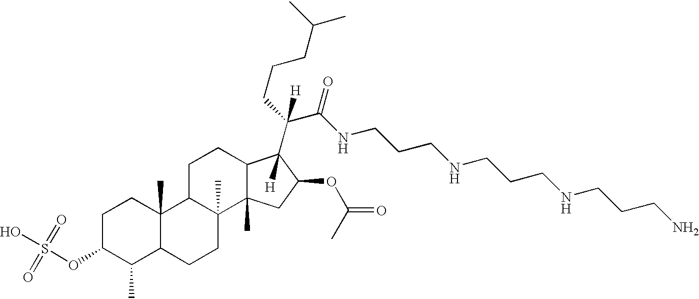 |

¹³C NMR (CD₃OD), δ/ppm: 177.5, 172.6, 81.8, 81.3, 52.5, 51.0, 50.7, 48.3, 47.9, 46.6, 46.0, 40.9, 40.5,
40.0, 39.9, 39.6, 38.2, 37.1, 36.1, 33.9, 32.7, 32.3, 30.4, 29.5, 29.1, 28.7, 28.7, 28.4, 27.0, 26.4, 23.1, 22.9,
21.6, 21.3, 21.2, 21.1, 17.8, 16.4

Example 81: Cream

| | |
|---|---|
| Compound 125 | 1 g |
| Petrolatum | 7.5 g |
| Liquid paraffin | 7.5 g |
| Spermaceti | 2.5 g |
| Sorbitane monopalmitate | 2.5 g |
| Polyoxyethylene sorbitane | |
| monopalmitate | 2.5 g |
| Water | 26.5 g |
| | 50 g |

Heat petrolatum, paraffin, spermaceti, sorbitane monopalmitate and polyoxyethylene sorbitane monopalmitate to 70° C. and slowly add water under continuous stirring. Continue stirring until the cream has cooled. Triturate compound 125 into the cream base and homogenise using a roller mill. Fill the cream into aluminium collapsible tubes.

Example 82: Ointment

| | |
|---|---|
| Compound 146 | 1 g |
| Liquid paraffin | 6.9 g |
| Cetanol | 0.2 g |
| Lanolin anhydrous | 2.3 g |
| Petrolatum | 39.6 g |
| | 50 g |

Melt paraffin, cetanol, lanolin and petrolatum at 70° C. After cooling to below 40° C. compound 149. Fill the ointment inot lacquered collapsible aluminium tubes.

Example 83: Capsules

| | |
|---|---|
| Compound 177 | 25 g |
| Microcrystalline cellulose | 14.5 g |
| Magnesium stearate | 0.5 g |
| | 40 g |

Pass the ingredients through a 60 mesh sieve and mix for 10 min. Fill the mixture into hard gelatine capsules using a capsule fill weight of 400 mg.

Example 84: Tablets

| | |
|---|---|
| Compound 125 | 25 g |
| Avicel TM | 12 g |
| STA-Rx 1500 | 12 g |
| Magnesium stearate | 1 g |
| | 50 g |

Compound 125 Avicel™ and STA-Rx are mixed together, sieved through a 0.7 mm sieve and thereafter mixed with magnesium stearate: The mixture is pressed into tablets each of 500 mg.

Example 85: Suspension

| | |
|---|---|
| Compound 180 | 1 g |
| Citric acid | 0.09 g |
| Sodium monohydrogenphosphate | 0.14 g |
| Sucrose | 5 g |
| Tween TM 80 | 0.01 g |
| Potassium sorbate | 0.04 g |
| Carboxymethylcellulose-Na | 0.1 g |
| Water | qs. to 100 ml suspension. |

The crystals are micronized and suspended in a solution of citric acid, sodium monohydrogen phosphate, sucrose, potassium sorbate and Tween™ 80 in 10 ml water, if necessary with slight warming. Carboxymethylcellulose-Na is dissolved in 4 ml boiling water. After cooling, it is added to the other ingredients. The suspension is homogenised in a blender and finally water is added to a total volume of 100 ml.

Example 86: Ointment

| | |
|---|---|
| A: 11-Compound 145 | 1 g |
| B: One of the compounds: hydrocortisone, triamcinolone or | 0.5 g |
| fluocinolone | |
| Liquid paraffin | 6.9 g |
| Cetanol | 0.2 g |
| Lanolin anhydrous | 2.3 g |
| Petrolatum | 39.1 g |
| | 50 g |

Melt paraffin, cetanol, lanolin and petrolatum at 70° C. After cooling to below 40° C., triturate A and B. Fill the ointment inot lacquered collapsible aluminium tubes.

Example 87: Ointment

| | |
|---|---|
| A: Compound 172 | 1.5 g |
| B: Tetracycline | 1.5 g |
| Liquid paraffin | 13.8 g |
| Cetanol | 0.4 g |
| Lanolin anhydrous | 4.6 g |
| Petrolatum | 78.2 g |
| | 100 g |

Melt paraffin, cetanol, lanolin and petrolatum at 70° C. After cooling to below 40° C., triturate A and B. Fill the ointment into lacquered collapsible aluminium tubes.

Example 88: Eye gel

| | |
|---|---|
| Compound 177 | 10 g |
| Benzalkonium chloride | 0.1 g |
| Carbomer | 5 g |
| Mannitol | 50 g |
| Sodium edetate | 0.5 g |
| Sodium hydroxide | q.s. |
| Sterile water | up to 100 g |

### REFERENCES

Abedel-Magdid, A. F., Carcon, K. G., Harris, B. D., Maryanoff, C. A., Shah, R. D., J. Org. Chem., 1996, 3849-3862.

Anelli, P. L., Lattuada, L:, Uggeri, F., 1998, Synth. Commun., 109-117.

Arigoni, D., von Daelne, W., Godtfredsen, W. O., Malera, A., Vangedal, S., 1964, Experimentia, 1-4.

Diassi, P. A., Bacso, I., Krakower, G. W., Ann Van Dine, H., 1966, Tetrahedron, 3459-3467.

Gaell, A. J., Blagbrough, I. S., 2000, Tetrahedron, 2449-2460.

Godtfredsen, W. O., Vangedal, S., 1962, Tetrahedron, 1029-1048.

Godtfredsen, W. O., Albrethsen, C., von Daehne, W., Tybring, L., Vangedal, S., 1965ₐ, Antimicrob. Agents Chemotherapy, 132-137.

Godtfredsen, W. O., von Daehne, W., Vangedal, S., Marquet, A., Arigoni, D., Melera, A., 1965_{b}, Tetrahedron, 3505-3530.

Godtfredsen, W. O., von Daehne, W., Tybring, L., Vangedal, S., 1966, J. Med. Chem., 15-22.

Goodnov, Jr., R., Konno, K., Niwa, M., Kallimopoulos, T., Bukownik, R., Lenares, D., Nakanishi, K., Tetrahedron, 1990, 3267-3286.

Hong-Seok Kim, H.-S., Bo-Seung Choi, B.-S., Kyung-Chan Kwon, K.-C., Sang-Ok Lee, S.-O, Hyun Jung Kwak, H. J., Cheol Hae Lee, C. H., 2000, Bioorg. Med. Chem., 2059-2065.

Karagiannis, G., Papaioannou, D., Eur. J. Org. Chem., 2000, 1841-1863.

Kikuchi, K., Bernard, E. M., Sadownik, A., Regen, S. L., Armstrong, D., 1997, Antimicrob. Agents . Chemoterap., 1433-1438.

Kinney, W. A., Zhang, X., Williams, J. I., Johnston, S., Michalak, R. S., Deshpande, M., Dostal, L., Rosazza, J. P. N., 2000, Org. Lett., 2921-2922.

Kuchers, A., Crove, S., Grayson, M. L., Hoy, J., in The use of antibiotics, 5.ed., Butterworths, Heinemann, Oxford, 1997.

Kuksa, V., Buchan, R., Kong Thoo Lin, P., 2000, Synthesis, 1189-1207.

Laurberg, M., Kristensen, O., Martemyanov, K., Gudkov, A.T., Nagaev, I., Hughes, D., Liljas, A., 2000, J. Mol. Biol., 593-603.

Moore, K. S., Wehrli, S., Roder, H., Rogers, M., Forrest, Jr., J. N., McCrirnmon, D., Zasloff, M., 1993, Proc. Natl. Acad. Sci. USA4, 1354-1358.

Pechulis, A. D., Bellevue III, F. H., Cioffi, C. L., Trapp, S. G., Fojtik, J. P., McKitty, A. A., Kinney, W. A., Frye, L. L., 1995, J. Org. Chem., 5121-5126.

Rao, M. N., Shinnar, A. E., Noecker, L. A., Chao, T. L., Feibush, B., Snyder, B., Sharkansky, I., Sarkahian, A, Zhang, X., Jones, S. R., Kinney, W. A., Zasloff, M., 2000, J. Nat. Prod., 631-635.

Sadownik, A., Deng, G., Janout, V., Regen, S. L., 1995, J. Am. Chem. Soc., 6138-6139.

Savage, P. B., Li, C., 2000, Exp. Opin. Invest. Drugs, 263-272.

Stromgaard, K., Brierley, M. J., Andersen, K., Slok, F. A., Mellor, I. R., Usherwood, P. N. R., Krogsgaard-Larsen, P., Jaroszewski, J. W., 1999, J. Med. Chem., 5224-5234.

von Daehne, W., Godtfredsen, W. O., Rasmussen, P., 1979, Adv. Appl. Microbiol., 95-145.

Weis, A. L., Bakos, T., Alferiev, I., Zhang, X., Shao, B., Kinney, W. A., 1999, Tetrahedron Lett., 4863-4864.

1. A compound of the general formula I 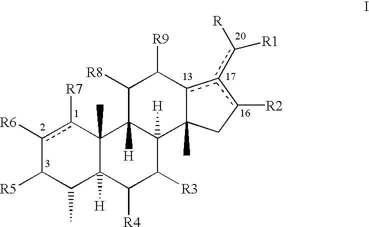
wherein
R₁ is hydrogen, halogen, CH₃, CH₂—OH, COOH, CH₂—OSO₃, CH₂—NH—(CH₂)ₐ—R₁₀, or C(═O)—NH—(CH₂)ₐ—R₁₀ wherein R₁₀ is —NH₂, —NH—(CH₂)_{b}—NH₂, —NH—(CH₂)_{b}—NH—(CH₂)_{c}—NH₂, —NH—(CH₂)_{b}—NH—(CH₂)_{c}—NH—(CH₂)_{d}—NH₂, —NH'(CH₂)_{b}—NH—(CH₂)_{c}—NH—(CH₂)_{d}—NH—(CH₂)ₑ—NH₂, —NH—(CH₂)_{b}—NH—(CH₂)_{c}—N—(CH₂)_{d}—NH—(CH₂)ₑ—NH—(CH₂)_{f}—NH₂, a saturated or unsaturated heterocyclic ring comprising 1 or 2 heteroatoms, or —NH—(CH₂)_{b}—R₁₁, wherein R₁₁ is a saturated or unsaturated heterocyclic ring comprising 1 or 2 heteroatoms, and a, b, c, d, e and fare the same or different and individually represent integers of from 1 to 5;
R₂ is hydrogen, halogen, —OH or —OR₁₂, wherein R₁₂ is SO₃, C₁₋₆ alkyl or C₁₋₆ acyl, —NH—(CH₂)ₐ—R₁₀;
R is hydrogen, halogen, a lipophilic group, —NH₂—(CH₂)ₐ—R₁₀ or CH₂—NH—(CH₂)ₐ—R₁₀;
R₄, R₅, R₆, R₇ and R₉ are the same or different and individually represent hydrogen, halogen, —OH, —OSO₃ or —NH—(CH₂)ₐ—R₁₀;
R₃ and R₈ are the same or different and individually represent hydrogen, halogen, —OH or OSO₃; and
the dotted lines between carbon atoms 1 and 2, 13 and 17, 16 and 17, and 17 and 20 indicate the presence of a single or double bond;
provided that at least one and not more than two of R, R₁, R₂, R₄, R₅, R₆, R₇ or R₉ is —NH—(CH₂)ₐ—R₁₀, CH₂—NH—(CH₂)ₐ—R₁₀ or C(═O)—NH—(CH₂)ₐ—R₁₀, and the others are hydrogen, —OH or —OSO₃, or (for R₂)—OR₁₂; and further provided that at least one and not more than four of R₂—R₉ are —OH or —OSO₃;
and pharmaceutically acceptable salts and esters thereof.

2. A compound according to claim 1 which has the general formula Ia 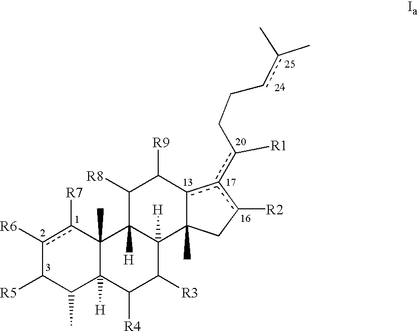
wherein
R₁ is CH₃, CH₂—NH—(CH₂)ₐ—R₁₀ or C(═O)—NH(CH₂)ₐ—R₁₀, wherein R₁₀ and a are as indicated above;
R₂ and R₅ are hydrogen, —OH or —OSO₃, or (for R₂)—OR₁₂, wherein R₁₂ is as indicated above; R₃, R₄, R₆, R₈ and R₉ are hydrogen, —OH or SO₃; and the dotted line between carbon atoms 1 and 2, 13 and 17, 16 and 17, 17 and 20, and 24 and 25 indicates the presence of a single or double bond;
provided that at least one and not more than four of R₂, R₃, R₄, R₅, R₆, R₈ and R₉ are —OH or OSO₃.

3. A compound according to claim 1 or 2, wherein a is 2 or 3.

4. A compound according to any of claims 1-3, wherein R₁₀ is —NH—(CH₂)_{b}—NH₂, wherein b has the meaning indicated in claim 1.

5. A compound according to any of claims 1-4, wherein b is 3 or 4.

6. A compound according to any of claims 1-3, wherein R₁₀ is —NH—(CH₂)_{b}—NH—(CH₂)_{c}—NH₂, wherein b and c are as indicated in claim 1.

7. A compound according to any of claims 1-6, wherein c is 2 or 3.

8. A compound according to any of claims 1-3, wherein R₁₀ is —NH—(CH₂)_{b}—NH—(CH₂)_{c}—NH—(CH₂)_{d}—MN₂, wherein b, c and d are as indicated in claim 1.

9. A compound according to any of claims 1-8, wherein d is 2, 3 or 4.

10. A compound according to any of claims 1-3, wherein R₁₀ is —NH—(CH₂)_{b}—NH—(CH₂)_{c}—NH—(CH₂)_{d}—NH—(CH₂)ₑ—NH₂, wherein b, c, d and e are as indicated in claim 1.

11. A compound according to any of claims 1-10, wherein e is 2, 3 or 4.

12. A compound according to claim 1 or 2, wherein R₁ is —NH—(CH₂)₃—NH—(CH₂)₄—NH—(CH₂)₃—NH₂, CH₂—NH—(CH₂)₃—NH—(CH₂)₄—NH—(CH₂)₃—NH₂ or C(═O)—NH—(CH₂)₃—NH—(CH₂)₄—NH—(CH₂)₃—NH₂.

13. A compound according to any of claims 1-12, wherein R₂ is —OR₁₂.

14. A compound according to claim 13, wherein R₁₂ is C₁₋₆ alkyl or C₁₋₆ acyl.

15. A compound according to claim 13, wherein R₁₂ is —NH(CH₂)ₐR₁₀, CH₂—NH—(CH₂)ₐ—R₁₀ or C(═O)—NH—(CH₂)ₐ—R₁₀.

16. A compound according to any of claims 1-15, wherein R₃, R₅ and/or R₈ are an —OH group.

17. A compound according to claim 1, wherein R is branched or straight C₁₋₁₀ alkyl, aryl, C₃₋₈ cycloalkyl, C₃₋₈ cycloalkenyl, aralkyl with 1-10 carbon atoms in the alkyl moiety, C₁₋₁₀ alkylaryl, C₁₋₁₀ alkyl-C₃₋₈ cycloalkyl, C₁₋₁₀ alkyl-C₃₋₈ cycloalkenyl, C₁₋₁₀ alkoxy or heteroaryl.

18. A compound according to claim 17, wherein R is as shown in formula Ia.

19. A compound according to any of claims 1-18 which is selected from the group consisting of
21-N-{3′-aminopropyl}-fusid-21-amide (Compound 101),
21-N-{2′-[(2′-aminoethyl)amino]ethyl}-fusid-21-amide (Compound 102),
21-N-{3′-[3′-aminoproyl)amino]propyl}-fusid-21-amide (Compound 103),
21-N-{3′-[(4′-aminobutyl)amino]propyl}-fusid-21-amide (Compound 104),
21-N-[2′-([3′-[(2′-aminoethyl)aminolpropyl}amino)ethyl]-fusid-21-amide (Compound 105),
21-N-[3′-({3′-[(3′-aminopropyl)amino]propyl}amino)propyl]-fusid-21-amide (Compound 106),
21-N-[3′-({4′-[(3′-aminopropyl)amino]butyl}amino)propyl]-fusid-21-amide (Compound 107),
21-N-[3′-({2′-[(3′-aminopropyl)amino]ethyl}aamino)propyl]-fusid-21-amide (Compound 108),
21-N-[4′-({3′-[(4′-aminobutyl)amino]propyl}amino)butyl]-fusid-21-amide (Compound 109),
21-N-{6′-[(6′-aminohexyl)amino]hexyl}-fusid-21-amide (Compound 110),
21-N-{8′-[(8′-aminooctyl)amino]octyl}-fusid-21-amide (Compound 111),
21-N-(2′-{[2′-({2′-[(2′-aminoethyl)amino]ethyl}amino)ethyl]amino}ethyl)-fusid-21-amide (Compound 112),
3-N-[3′-({3′-[(3′-aminopropyl)amino]propyl}amino)propyl]-fusidic acid (Compound 113)
3-N-[2′-({3′-[(2′-aminoethyl)aminojpropyl}amino)ethyl]-fusidic acid (Compound 114)
3-N-[3′-({2′-[(3′-aminopropyl)amino]ethyl}amino)propyl]fusidic acid (Compound 115)
21-N-{2′-({2′-[(2′-aminoethyl)amino]ethyl}amino)ethyl)amino}-17R,20S,24,25-tetrahydrofusid-21-amide (Compound 116),
21-N-{2′-[(2′-aminoethyl)amino]ethyl}-17R,20S,24,25-tetrahydrofusid-21-amide (Compound 117),
21-N-{6′-aminohexyl}-17R,20S,24,25-tetrahydrofusid-21-amide (Compound 118),
21-N-{3′-aminopropyl}-17R,20S,24,25-tetrahydrofusid-21-amide (Compound 119),
21-N-{3′-[3′-aminoproyl)amino]propyl}-17R,20S,24,25-tetrahydrofusid-21-amide (Compound 120),
21-N-{4′-[(3′-aminopropyl)amino]butyl}-17R,20S,24,25-tetrahydrofusid-21-amide (Compound 121),
21-N-A[2′-({3′-[(2′-aminoethyl)amino]propyll amino)ethyl]-17R,20S,24,25-tetrahydrofiusid-21-amide (Compound 122),
21-N-[4′-({3′-[(4′-aminobutyl)amino]propyl}amino)butyl]-17R,20S,24,25-tetrahydrofusid-21-amide (Compound 123),
21-N-[3′-({2′-[(3′-aminopropyl)amino]ethyl}amino)propyl]-17R,20S,24,25-tetrahydrofusid-21-amide (Compound 124),
21-N-[3′-({4′-[(3′-aminopropyl)amino]butyl}amino)propyl]-17R,20S,24,25-tetrahydrofusid-21-amide (Compound 125),
21-N-{6′-[(6′-aminohexyl)amino]hexyl}-17R,20S,24,25-tetrahydrofusid-21-amide (Compound 126),
21-N-{8′-[(8′-aminooctyl)amino]octyl}-17R,20S,24,25-tetrahydrofusid-21-amide (Compound 127),
21-N-[3′-({3′-[(3′-aminopropyl)aminolpropyl}amino)propyl]-17R,20S,24,25-tetrahydrofusid-21-amide (Compound 128),
21-N-(2′-{[2′-({2′-[(2′-aminoethyl)amino]ethyl}amino)ethyl]amino}ethyl)-17R,20S,24,25-tetrahydrofusid-21-amide (Compound 129),
21-N-{6′-aminohexyl}-17R,20S,24,25-tetrahydrofusid-21-amide (Compound 130),
21-N-{3′-[3′-aminoproyl)amino]propyl}-17R,20S,24,25-tetrahydrofusid-21-amide (Compound 131),
21-N-{2′-[(2′-aminoethyl)amino]ethyl}-17R,20S,24,25-tetrahydrofusid-21-amide (Compound 132),
21-N-{2′-({3′-[(2′-aminoethyl)amino]propyl}amino)ethyl}-17R,20S,24,25-tetrahydrofusid-21-amide (Compound 133),
21-N-{2′-({2′-[(2′-aminoethyl)amino]ethyl}amino)ethyl)amino}-17R,20S,24,25-tetrahydrofusid-21-amide (Compound 134),
21-N-[3′-({4′-[(3′-aminopropyl)amino]butyl}amino)propyl]-17R,20S,24,25-tetrahydrofusid-21-amide (Compound 135),
21-N-(2′-{[2′-({2′-[(2′-aminoethyl)amino]ethyl}amino)ethyl]amino}ethyl)-17R,20S,24,25-tetrahydrofusid-21-amide (Compound 136),
21-N-{4′-[(3′-aminopropyl)amino]butyl}-17R,20S,24,25-tetrahydrofusid-21-amide (Compound 137),
21-N-{[4′-({3′-[(4′-aminobutyl)amino]propyl}amino)butyl]}-17R,20S,24,25-tetrahydrofusid-21-amide (Compound 138),
21-N-[3′-({3′-[(3′-aniinopropyl)amino]propyl}amino)propyl]-16(17)-en-17,20,24,25-tetrahydrofusidan-21-carboxamide (Compound 139),
21-N-{6′-aminohexyl}-16-desacetoxy-17R,20S,24,25-tetrahydrofusid-21-amide (Compound 140),
21-N-{6′-aminohexyl}-16-desacetoxy-17R,20S,24,25-tetrahydrofusid-21-amide (Compound 141), (C-20 epimer of Compound 140),
21-N-{2′-[(2′-aminoethyl)amino]ethyl}-16-desacetoxy-17R,20S,24,25-tetrahydrofusid-21-amide (Compound 142),
21-N-{3′-[3′-aminoproyl)amino]propyl}-16-desacetoxy-17R,20S,24,25-tetrahydrofusid-21-amide (Compound 143),
21-N-{3′-[(4′-aminobutyl)amino]propyl}-16-desacetoxy-17R,20S,24,25-tetrahydrofusid-21-amide (Compound 144),
21-N-{[3′-({3′-[(3′-aminopropyl)amino]propyl}amino)propyl]}-16-desacetoxy-17R,20S,24,25-tetrahydrofusid-21-amide (Compound 145),
21-N-{[3′-({4′-[(3′-aminopropyl)amino]butyl}amino)propyl]}-16-desacetoxy-17R,20S,24,25-tetrahydrofusid-21-amide (Compound 146),
21-N-(2′-{[2′-({2′-[(2′-aminoethyl)amino]ethyl}amino)ethyl]amino) ethyl)-16-desacetoxy-17R,20S,24,25-tetrahydrofusid-21-amide (Compound 147),
21-N-{2′-({2′-[(2′-aminoethyl)amino]ethyl}amino)ethyl)aminol-16-desacetoxy-17R,20S,24,25-tetrahydrofusid-21-amide (Compound 148),
21-N-[2′-({3′-[(2′-aminoethyl)amino]propyl}amino)ethyl]-16-desacetoxy-17R,20S,24,25-tetrahydrofusid-21-amide (Compound 149),
21-N-{6′-aminohexyl}-11-desoxy-16-desacetoxy-17S,20,24,25-tetrahydrofusid-21-amide (Compound 150),
21-N-{3′-[3′-aminoproyl)amino]propyl}-11-desoxy-16-desacetoxy-17S,20,24,25-tetrahydrofusid-21-amide (Compound 151),
21-N-[2′-({3′-[(2′-aminoethyl)amino]propyl}amino)ethyl]-11-desoxy-16-desacetoxy-17S,20,24,25-tetrahydrofusid-21-amide (Compound 152),
21-N-[3′-({2′-[(3′-aminopropyl)amino]ethyllamino)propyl]-11-desoxy-16-desacetoxy-17S,20,24,25-tetrahydrofusid-21-amide (Compound 153),
21-N-{4′-[(3′-aminopropyl)amino]butyl}-11-desoxy-16-desacetoxy-17S,20,24,25-tetrahydrofusid-21-amide (Compound 154),
21-N-[3′-({3′-[(3′-aminopropyl)amino]propyl}amino)propyl]-11-desoxy-16-desacetoxy-17S,20,24,25-tetrahydrofusid-21-aride (Compound 155),
21-N-[3′-({4′-[(3′-aminopropyl)amino]butyl}amino)propyl]-11-desoxy-16-desacetoxy-17S,20,24,25-tetrahydrofusid-21-amide (Compound 156),
21-N—[4′-({3′-[(4′-aminobutyl)amino]propyl}amino)butyl]-11-desoxy-16-desacetoxy-17S,20,24,25-tetrahydrofusid-21-amide (Compound 157),
21-N-{6′-aminohexyl}-3β-desacetoxy-17R,20S,24,25-tetrahydrofusid-21-amide (Compound 158),
21-N-{2′-[(2′-aminoethyl)amino]ethyl}-3β-desacetoxy-17R,20S,24,25-tetrahydrofusid-21-amide (Compound 159),
21-N-{3′-[3′-aminoproyl)amino]propyl}-3β-desacetoxy-17R,20S,24,25-tetrahydrofusid-21-amide (Compound 160),
21-N-{3′-[(4′-aminobutyl)amino]propyl}-3β-desacetoxy-17R,20S,24,25-tetrahydrofusid-21-amide (Compound 161),
21-N-[2′-({3′-[(2′-aminoethyl)amino]propyl}amino)ethyl]-3β-desacetoxy-17R,20S,24,25-tetrahydrofusid-21-amide (Compound 162),
21-N-[3′-({3′-[(3′-aminopropyl)amino]propyl}amino)propyl]-3β-desacetoxy-17R,20S,24,25-tetrahydrofusid-21-amide (Compound 163),
21-N-(2′-{[2′-((2′-[(2′-aminoethyl)amino]ethyl) amino)ethyl]aminolethyl)-3β-desacetoxy-17R,20S,24,25-tetrahydrofusid-21-amide (Compound 164),
21-N-[4′-({3′-[(4′-aminobutyl)amino]propyl) amino)butyl]-3β-desacetoxy-17R,20S,24,25-tetrahydrofusid-21-amide (Compound 165),
21-N-[3′-({4′-[(3′-aminopropyl)amino]butyl}amino)propyl]-3β-desacetoxy-17R,20S,24,25-tetrahydrofusid-21-amide (Compound 166),
21-N-{6′-aminohexyl) -3-OAc-17R,20S,24,25-tetrahydrofusid-21-amide (Compound 167),
21-N-{3′-[3′-aminoproyl)amino]propyl}-3-OAc-17R,20S,24,25-tetrahydrofusid-21-amide (Compound 168),
21-N-{3′-[(4′-aminobutyl)amino]propyl}-3-OAc-17R,20S,24,25-tetrahydrofusid-21-amide (Compound 169),
21-N-[2′-({3′-[(2′-aminoethyl)amino]propyl}amino)ethyll-3-OAc-17R,20S,24,25-tetrahydrofusid-21-amide (Compound 170),
21-N-[3′-({2′-[(3′-aminopropyl)amino]ethyl}amino)propyl]-3-OAc-17R,20S,24,25-tetrahydrofusid-21-amide (Compound 171),
21-N-[3′-({3′-[(3′-aminopropyl)amino]propyl}amino)propyl]-3-OAc-17R,20S,24,25-tetrahydrofusid-21-amide (Compound 172),
21-N-[3′-({4′-[(3′-aminopropyl)amino]butyl}amino)propyl]-3-OAc-17R,20S,24,25-tetrahydrofusid-21-amide (Compound 173),
21-N-{3′-[3′-aminoproyl)amino]propyl}-3-OSO₃-11-esoxy-17,20,24,25-tetrahydrofusid-21-amide (Compound 174),
21-N-[2′-({3′-[(2′-aminoethyl)amino]propyl}amino)ethyl]-3-OSO₃-11-desoxy-17,20,24,25-tetrahydrofusid-21-amide (Compound 175),
21 N-{3′-[(4′-aminobutyl)amino]propyl3-3-OSO₃-11-desoxy-17,20,24,25-tetrahydrofusid-21-amide (Compound 176),
21A-N-[3′-({4′-[(3′-aminopropyl)amino]butyl}amino)propyl]-3-OSO₃-1l-desoxy-17,20,24,25-tetrahydrofusid-21-amide (Compound 177),
21-N-(2′-{[2′-({2′-[(2′-aminoethyl)amino]ethyl}amino)ethyl]amino}ethyl)-3-OSO₃-11-desoxy-17,20,24,25-tetrahydrofusid-21-amide (Compound 178),
21-N-[3′-({2′-[(3′-aminopropyl)amino]ethyl}amino)propyl]-3-OSO₃-11-desoxy-17,20,24,25-tetrahydrofusid-21-amide (Compound 179),
21-N-[4′-({3′-[(4′-aminobutyl)amino]propyl}amino)butyl]-3-OS03-1l-desoxy-17,20,24,25-tetrahydrofusid-21-amide (Compound 180).

20. A pharmaceutical composition comprising a compound according to any of claims 1-19, optionally together with a pharmaceutically acceptable excipient or diluent, and optionally together with another therapeutically active agent.

21. A composition according to claim 20 which is in the form of a topical formulation.

22. A composition according to claim 21 which is a cream, ointment, salve or lotion.

23. A composition according to any of claims 20-23 wherein said other agent is selected from the group consisting of penicillins, cephalosporins, tetracyclins, rifamycins, erythromycins, lincomycin, clindamycin, fluoroquinolones, hydrocortisone and triamcinolone.

24. A compound according to any of claims 1-19 for use as a medicament.

25. A compound according to claim 24 for use as an antimicrobial agent.

26. A compound according to claim 25 for use as an antibacterial agent.

27. Use of a compound according to any of claims 1-19 for the preparation of a medicament for the prevention or treatment of infection.

28. The use according to claim 27 for the prevention or treatment of bacterial infection.

29. The use according to claim 27 or 28, wherein said compound is combined with one or more other therapeutically active agents.

30. The use according to any of claims 27-29, wherein the medicament is intended for topical administration.

30. A method of preventing or treating infection, the method comprising administering to a patient in need thereof an effective amount of a compound according to any of claims 1-19.

31. A method according to claim 30, wherein said infection is a bacterial infection.

32. A method according to claim 30 or 31, wherein said compound is administered topically.

33. A method according to any of claims 30-32, wherein said compound is administered together with one or more other therapeutically active agents.

34. A method according to claim 33 wherein said other therapeutically active agent is selected fro the group consisting of penicillins, cephalosporins, tetracyclins, rifamycins, erythromycins, lincomycin, clindamycin, fluoroquinolones, hydrocortisone and triamcinolone
